(19) United States
(12) Patent Application Publication
(10) Pub. No.: US 2007/0203139 A1
(43) Pub. Date: Aug. 30, 2007

(54) Aryl Glycinamide Derivatives And Their Use As Nk1 Antagonists And Serotonin Reuptake Inhibitors

(75) Inventors: Peter Bernstein, Rose Valley, PA (US); Cathy Dantzman, Wilmington, DE (US); William Palmer, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 10/599,823

(22) PCT Filed: Apr. 6, 2005
(86) PCT No.: PCT/SE05/00501

§ 371 Date: Oct. 11, 2006

(30) Foreign Application Priority Data
Apr. 14, 2004 (SE) 0400969-2
Apr. 14, 2004 (SE) 0400967-6

(51) Int. Cl. A61K 31/5375; A61K 31/495; A61K 31/445; A61K 31/40; A61K 31/277

(52) U.S. Cl. 514/237.8; 514/252.12; 514/317; 514/408; 514/521; 514/616; 544/159; 544/399; 546/229; 548/561; 558/410; 564/152; 514/319

(57) ABSTRACT

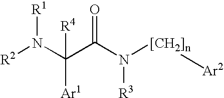

Compounds of the following Formula (I) wherein R¹ and R² are independently selected from alkyl or alkenyl or from a heterocyclic ring together with the N to which they are bound, n is 0-2, Ar¹ is (substituted) phenyl and Ar¹ is (substituted) phenyl, naphthyl or tetralin, further as defined in the specification, in vivo-hydrolysable precursors and pharmaceutically acceptable salts thereof, the use in therapy and pharmaceutical compositions and methods of treatment using the same. The compounds are neurokinin 1 (NK₁) receptor antagonists and/or serotonin reuptake inhibitors, with medical indications for depression, anxiety disorders and other conditions.

### FIELD OF THE INVENTION

This invention relates to the treatment of diseases in which Substance P is implicated, for example, in the treatment of disorders or conditions such as depression, generalized anxiety disorder, phobias, posttraumatic stress syndrome, avoidant personality disorder, obsessive-compulsive disorder, panic disorder, cerebellar ataxia, gastrointestinal tract disorders, negative symptoms of schizophrenia, premenstrual syndrome, stress incontinence.

### BACKGROUND

The mammalian neurokinins are peptide neurotransmitters found in the peripheral and central nervous systems. The three principal neurokinins are Substance P (SP), Neurokinin A (NKA) and Neurokinin B (NKB). N-terminally extended forms of at least NKA are known. Three receptor types are known for the principal neurokinins. Based upon their relative selectivities for the neurokinins SP, NKA and NKB, the receptors are classified as neurokinin 1 (NK₁), neurokinin 2 (NK₂) and neurokinin 3 (NK₃) receptors, respectively. In the periphery, SP and NKA are localized in C-afferent sensory neurons, which neurons are characterized by non-myelinated nerve endings known as C-fibers, and are released by selective depolarization of these neurons, or selective stimulation of the C-fibers. C-Fibers are located in the airway epithelium, and the tachykinins are known to cause profound effects which clearly parallel many of the symptoms observed in asthmatics. The effects of release or introduction of tachykinins in mammalian airways include bronchoconstriction, increased microvascular permeability, vasodilation, increased mucus secretion and activation of mast cells. Neurokinin antagonists that interact with NK₁, NK₂ and NK₃ receptors, having different chemical structures have been described. Particularly international publications WO 98/07722, WO 96/39383 and WO 98/25617, and regional publications EP 428434, EP 474561, EP 515240 and EP 559538 disclose the preparation of a variety of chemical structures.

NK₁ activity is also implicated in depression and anxiety, mice with genetically altered NK₁ receptors have decreased anxiety related behavior (Santarelli, L., et. al., Proc. Nat. Acad. Sci. (2001), 98, 1912) and NK₁ antagonists have been reported to be effective in an animal model of depression (Papp, M., et. al., Behav. Brain Res. (2000), 115, 19).

A selective Substance P antagonist has been asserted to be efficacious and safe for the treatment of major depression (Kramer, M. S. et al., Neuropsychopharmacology (2004) 29, 385-392.

### DESCRIPTION OF THE INVENTION

The present invention encompasses compounds having neurokinin 1 (“NK₁”) antagonist activity. Aryl glycine compounds of the invention are those in accord with formula I 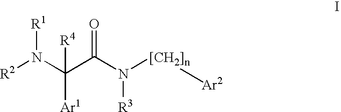
wherein:

R¹ and R² are independently selected from C₁₋₆alkyl or C₁₋₆alkenyl, or together with the N to which they are bound, form a heterocycle having 4, 5, 6, 7 or 8 atoms or such a heterocycle substituted with moieties independently selected from hydrogen, halogen, C₁₋₄alkyl, C₁₋₄alkoxy or C₁₋₄alkyl substituted with 1, 2 or 3 halo moieties, amino, or amino substituted with C₁₋₄alkyl, C₁₋₄alkoxy or C₁₋₄alkyl substituted with 1, 2 or 3 halo moieties;

R³ is C₁₋₆alkyl;

R⁴ is hydrogen;

n is 0, 1 or 2;

Ar¹ is phenyl or phenyl substituted with moieties independently selected from hydrogen, halogen, C₁₋₄alkyl, C₁₋₄alkoxy or C₁₋₄alkyl substituted with 1, 2 or 3 halo moieties, and

Ar² phenyl, naphthyl, tetralin, or phenyl, naphthyl or tetralin substituted with moieties independently selected from hydrogen, halogen, cyano, nitro, C₁₋₄alkyl, C₁₋₄alkoxy or C₁₋₄alkyl substituted with 1, 2 or 3 halo moieties.

The invention also encompasses enantiomers, stereoisomers, in vivo-hydrolysable precursors and pharmaceutically-acceptable salts of the compounds, pharmaceutical compositions and formulations containing them, methods of using them to treat diseases and conditions either alone or in combination with other therapeutically-active compounds or substances, processes and intermediates used to prepare them, uses of them as medicaments, uses of them in the manufacture of medicaments and uses of them for diagnostic and analytic purposes.

Particular compounds of the invention are those in accord with formula II 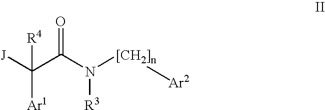
wherein:

J is —NR¹R² as defined heretofore, or J is selected from moieties of formula III, IV or V, 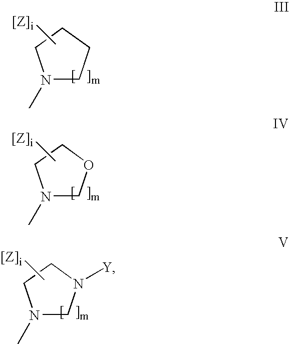
wherein:
when J is —NR¹R²,
R¹ and R² are independently selected from H, C₁₋₆alkyl, C₁₋₆alkenyl, C₁₋₆alkanoyl, —CH₂—C(═O)—O—R⁹ or heterocycle,
   wherein any such C₁₋₆alkyl, C₁₋₆alkenyl, C₁₋₆alkanoyl, or heterocycle moiety may be substituted with 1, 2 or 3 halo moieties, amino, or amino substituted with C₁₋₄alkyl, C₁₋₄alkoxy or C₁₋₄alkyl substituted with 1, 2 or 3 halo moieties, and
   R⁹ is selected from hydrogen or C₁₋₆alkyl;
or —(CH₂)ₖX,
   where X is selected from —OH, —OR⁵, —C(═O)R⁵ or —NR⁵R⁶ and k is 0, 1, 2, 3 or 4,
      wherein R⁵ and R⁶ are independently selected from H, C₁₋₆alkyl, C₁₋₆alkoxy,
      C₁₋₆alkoxymethylene or C₁₋₆alkenyl,
         where any such C₁₋₆alkyl, C₁₋₆alkoxy, C₁₋₆alkoxymethylene or
         C₁₋₆alkenyl may have 1, 2 or 3 halogen substituents,
      or R⁵ and R⁶ together with a N to which they are bound form a heterocycle having 4, 5, 6 or 7 atoms or such a heterocycle substituted with moieties independently selected from halogen, C₁₋₄alkyl, C₁₋₄alkoxy or C₁₋₆alkanoyl, or C₁₋₄alkyl or C₁₋₆alkanoyl substituted with 1, 2 or 3 halo moieties, amino, or amino substituted with C₁₋₄alkyl, C₁₋₄alkoxy or C₁₋₄alkyl, substituted with 0, 1, 2 or 3 halo moieties, and
with the proviso that R¹ and R² are not both hydrogen;
   when J is a moiety of formula III, m is 0, 1 or 2;
   when J is a moiety of formula IV, m is 2 or 3;
   when J is a moiety of formula V, m is 2 or 3 and Y is selected from H, C₁₋₆alkyl, C₁₋₆alkenyl, C₁₋₆alkanoyl or C₁₋₆alkoxycarbonyl where any such C₁₋₆alkyl, C₁₋₆alkenyl, C₁₋₆alkanoyl or C₁₋₆alkoxycarbonyl may have 1, 2 or 3 halogen substituents;
wherein for any moiety of formula III, IV or V, Z is C₁₋₆alkyl, —NR⁷R⁸, or halogen, and i is 0, 1 or2
   wherein R⁷ and R⁸ are independently selected from H, C₁₋₆alkyl C₁₋₆alkenyl or —(CH₂)ₖX, where X is selected from H, —OH, —OR⁵, —C(═O)R⁵ or —NR⁵R⁶, or R⁷ and R⁸ together with the N to which they are bound, form a moiety of formula VI, VII, VIII or IX, 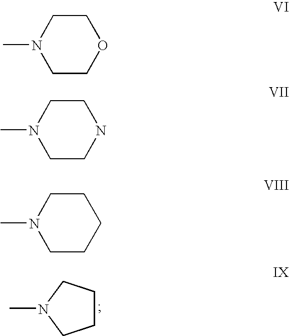
wherein any said moiety of formula VI, VII, VIII or IX may be substituted with 1, 2 or 3 moieties selected from C₁₋₄alkyl, halogen or ═O;

Ar¹ is phenyl or phenyl substituted with moieties independently selected from hydrogen, halogen, C₁₋₄alkyl, C₁₋₄alkoxy or C₁₋₄alkyl substituted with 1, 2 or 3 halo moieties; and

Ar² is phenyl, naphthyl, tetralin, or phenyl, naphthyl or tetralin substituted with moieties independently selected from hydrogen, halogen, cyano, nitro, C₁₋₄alkyl, C₁₋₄alkoxy or C₁₋₄alkyl substituted with 1, 2 or 3 halo moieties;
with the proviso that when J is a moiety of formula V, Ar² is not phenyl, in vivo-hydrolysable precursors thereof, and pharmaceutically-acceptable salts thereof.

Particular compounds of the invention are those of the examples herein.

Another aspect of the invention is pharmaceutically-acceptable salts of a compounds as described herein made with an inorganic or organic acid which affords a physiologically-acceptable anion.

Particular pharmaceutically-acceptable salts of compounds of the invention are those wherein the inorganic or organic acid is selected from hydrochloric, hydrobromic, sulfuric, phosphoric, methanesulfonic, sulfamic, para-toluenesulfonic, acetic, citric, lactic, tartaric, malonic, fumaric, ethanesulfonic, benzenesulfonic, cyclohexylsulfamic, salicyclic and quinic acids.

Another aspect of the invention is a pharmaceutical composition comprising a compound of the invention or an in vivo-hydrolysable precursor or a pharmaceutically-acceptable salt thereof and a pharmaceutically-acceptable carrier.

Yet another aspect of the invention is a method of treating a disease condition wherein antagonism of NK₁ receptors is beneficial which method comprises administering to a warm-blooded animal an effective amount of a compound of the invention or an in vivo-hydrolysable precursor or a pharmaceutically-acceptable salt thereof. Still another aspect of the invention is the use of a compound of the invention or an in vivo-hydrolysable precursor or a pharmaceutically-acceptable salt thereof in the preparation of a medicament for use in a disease condition wherein antagonism of the NK₁ receptors is beneficial.

A further aspect of the invention is a method for treating a disorder or condition selected from depression in cancer patients, depression in Parkinson's patients, postmyocardial infarction depression, subsyndromal symptomatic depression, depression in infertile women, pediatric depression, major depression, single episode depression, recurrent depression, child-abuse induced depression, post-partum depression, generalized anxiety disorder, agoraphobia, social phobia, simple phobias, posttraumatic stress syndrome, avoidant personality disorder, obsessive-compulsive disorder, panic disorder, dementia, hyperprolactinaemia, cerebellar ataxia, gastrointestinal tract disorders, negative symptoms of schizophrenia, premenstrual syndrome and stress incontinence, wherein antagonism of the NK₁ receptors is beneficial, comprising administering an effective amount of a compound of the invention or a pharmaceutically-acceptable salt thereof effective in treating such disorder or condition.

In a particular aspect of the invention the method for treating a disorder or condition mentioned herein, comprises administering a compound of the invention in combination with a pharmaceutically-acceptable carrier.

Compounds in accord with formula I and their in vivo-hydrolysable precursors or a pharmaceutically-acceptable salts may be made by processes as described and exemplified herein and by processes similar thereto and by processes known in the chemical art. If not commercially available, starting materials for these processes may be made by procedures which are selected from the chemical art using techniques which are similar or analogous to the synthesis of known compounds.

Pharmaceutically-acceptable salts may be prepared from the corresponding acid in a conventional manner. Non-pharmaceutically-acceptable salts may be useful as intermediates and as such are another aspect of the present invention.

It is well known in the art how to prepare optically-active forms (for example, by resolution of the racemic form or by synthesis from optically-active starting materials) and all optically active forms, enantiomers are compounds of this invention. Further, the mixture of enantiomers can have neurokinin activity and either of the pure enantiomers can have neurokinin activity.

The following biological test methods, data and Examples serve to illustrate and further describe the invention.

The utility of a compound of the invention or an in vivo-hydrolysable precursor or a pharmaceutically-acceptable salt thereof (hereinafter, collectively referred to as a “Compound”) may be demonstrated by standard tests and clinical studies, including those disclosed in the publications described below.

Biological Assays:

NK₁ FLIPR Assay Using Fluo-4 Dye:

FLIPR assays are performed with a device marketed by Molecular Devices, Inc., designed to precisely measure cellular fluorescence in a high throughput whole-cell assay. (Schroeder et. al., J. Biomolecular Screening, 1(2), p 75-80, 1996).

Compounds were evaluated for potency in blocking the response of U373 cells to the NK₁ receptor agonist Acetyl-[Arg⁶, Sar⁹, Met(0₂)¹¹]-Substance P (ASMSP) using a FLIPR instrument.

U373 cells were loaded with Fluo-4 dye (Molecular Probes) for 45 min at 37° C. and exposed to graded concentrations of compounds for 15 min at room temperature before being challenged with 10 nM-12 nM ASMSP (an approximately EC₈₀ concentration). Responses were measured as the peak relative fluorescence after agonist addition. pIC₅₀s were calculated from eleven-point concentration-response curves for each compound.

Reagents:

Cell Culture Medium:

| | |
|---|---|
| Eagle's MEM with Earle's salts and 1-glutamine | Cellgro 10-010-CV |
| (500 mL) | |
| Non-essential amino acids, 100× (5 mL) | Cellgro 25-025-CI |
| Sodium pyruvate, 100 mM (5 mL) | Cellgro 25-000-CI |
| L-Glutamine, 200 mM (5 mL) | Cellgro 25-005-CI |
| FBS (50 mL) | Cellgro 35-010-CV |

Cell Harvesting Reagents:

| | |
|---|---|
| DPBS, 1× without Ca⁺⁺ & Mg⁺⁺ | Cellgro 21-031-CV |
| 1× Trypsin -EDTA (0.5% Trypsin, 0.53% | Cellgro 25-052-CI |
| EDTA-4Na) | |

Cell Plating Medium:

| | |
|---|---|
| UltraCULTURE | BioWhittaker 12-725F |
| L-Glutamine, 200 mM (5 mL/500 mL) | Cellgro 25-005-CI |

Working Buffer:

| | |
|---|---|
| 10× Hank's balanced salt solution (100 mL/L) | Gibco 14065-056 |
| HEPES buffer 1 M (15 mL/L, [final] 15 mM) | Cellgro 25-060-CI |
| Probenecid (0.71 g dissolved in 6 mL 1 M NaOH | Sigma P-8761 |
| for 1 L, [final] 2.5 mM) | |
| DDH₂0 to 1 L, adjust pH to 7.4 with NaOH | |

Dye Solution:
Fluo-4, AM dye, Molecular Probes F-14201. 50 μg lyophilized dye is dissolved in 23 μl DMSO plus 23 μL Pluronic F-127 (Molecular Probes P-3000). The 46 μL of solubilized fluo-4 dye is then added to 10 mL of working buffer solution to provide a working dye concentration of 5 μM. Each 10 mL of diluted dye is sufficient for a 384-well-plate of cells at 25 μL per well.

Agonist:
Acetyl-[Arg⁶, Sar⁹, Met(O₂)¹¹]-Substance P (ASMSP)
Stock solution of 3.33×10⁻² M. Dissolve 100 mg in 3.05 mL DMSO and store in aliquots at 4° C.

Miscellaneous:
DMSO (to dissolve compounds and for tip wash)
   Cell Culture and Plating Procedures:

U373 cells were grown in cell culture medium described above (30 mL per T-150 flask) and harvested when confluent as follows. Medium was removed by aspiration and cells were washed 1× with 12 mL DPBS, without Ca++ and Mg++. The DPBS was aspirated and replaced with 3 mL trypsin EDTA. The cells plus trypsin/EDTA were incubated about 2 min at room temperature, until the cells detached from the flask. The harvesting reaction was quenched by addition of 9 mL culture medium and cells were resuspended by trituration.

Cells were passaged at a transfer density of 1:4 every four days. For experiments, cells were counted, pelleted by centrifugation at 400×g for 5 min and resuspended in cell plating medium at a density of 480,000 cells/mL. 25 μL of this cell suspension was added to each well of a black-walled 384-well plate (Falcon Microtest, 35 3962) using a Labsystems Multidrop 384 to give 12,000 cells per well. Plates were incubated at 37° C. overnight (minimum 15 h, maximum 23 h) before use.

Compound and Agonist Preparation:

Compounds were dissolved in DMSO at a concentration of 10 mM and 120 μL of these solutions were transferred to the first well (column 1) of each row of a 96-well, round-bottomed, polypropylene storage plate (Costar 3365). Compounds on two such plates were then serially diluted simultaneously in DMSO using a Biomek 2000. 4 μL of each dilution was transferred to a deep well plate (Beckman Coulter 267006) which had been prepared previously to contain 400 μL of freshly made working buffer in each well. Concentrations resulting from this procedure are shown in Table 1. The final compound concentrations in the assay span 11 points, between 10 μM and 0.1 nM, in half-log increments.

The contents of the wells were mixed, and 45 μL of each dilution were transferred—in duplicate—to a 384-well polypropylene compound loading plate (Fisher 12-565-507) so that the 384-well plate contained duplicates of each of the compounds from both 96-well plates over the concentration ranges. Columns 23 and 24 of the plate contain no compound and serve as controls. Wells A-N in columns 23 and 24 were loaded with agonist only and therefore represent the maximal response. Wells O-P in columns 23 and 24 were loaded with only buffer, no agonist, and therefore represent the minimum response.

An ASMSP agonist loading plate was made by taking stock concentration of ASMSP and diluting in working buffer to give a concentration of 3.3×10⁻⁸ M. 45 μL of this solution were transferred to all wells of a 384-well polypropylene agonist loading plate (Fisher 12-565-507) except wells 023, 024, P23 & P24 which contained buffer alone and served as unstimulated controls.

Dye Loading Cells and Adding Compound:

For each 384-well assay plate of cells, 10 mL of diluted Fluo-4 dye was prepared as stated above in the methods/reagents section. First, each 384-well cell plate was washed once with working buffer on a CCS Packard plate washer. Any remaining post-wash buffer in the wells was removed by hand and 25 μL per well of Fluo-4 dye was added using a Labsystems Multidrop 384. The cell plate was returned to a 37° C. incubator for 45 min to allow the dye to permeate the cells. After 45 min of dye loading, the cell plates were washed twice with working buffer, leaving a 30 μL volume of buffer in each well. 5 μL of compound dilutions were transferred from the compound plate to the cell plate using a PlateMate. Assay plates were incubated in the presence of compound for 15 min at room temperature in the dark, and then loaded onto FLIPR.

Recording Responses in FLIPR:

After the 15 min compound pre-incubation, the plates were loaded onto the FLIPR instrument, 15 μL of ASMSP agonist was added and the cellular response to the agonist was recorded for 90 seconds. The response is measured as the peak relative fluorescence after agonist addition.

Data analysis:

Results contained in the stat files generated by FLIPR were pasted into an Excel analysis template and, after outliers were excluded, IC₅₀ values were calculated within the template using XLfit. Individual IC₅₀ values were reported, along with pIC₅₀. When the two IC₅₀'s obtained for a compound differed by more than 3-fold that compound was assayed one or two more times to re-determine the value.

Results:

Ki values obtained in the SERT assay for compounds of the invention ranged from less than 2 nM to about 180 nM. IC₅₀ values obtained in the FLIPR assay for compounds of the invention ranged from about 70 nM to about 2 μM.

### EXAMPLES

The invention is illustrated by, but not limited to, the following examples in which descriptions, where applicable and unless otherwise stated, the following terms, abbreviations and conditions are used:

aq., aqueous; atm, atmospheric pressure; BOC, 1,1-dimethylethoxycarbonyl, ACN, acetonitrile; DCM, dichloromethane; DMR, N,N-dimethylformamide; DMSO, dimethyl sulfoxide; EtOH, ethanol; Et₂O, diethyl ether; EtOAc, ethyl acetate; h, hour(s); HPLC, high performance liquid chromatography; HOBT, 1-hydroxybenzotriazole; MeOH, methanol; min, minutes; MS, mass spectrum; NMR, nuclear magnetic resonance; psi, pounds per square inch; RT, room temperature; sat., saturated; TEA, triethylamine; TFA, trifluoroacetic acid; THF, tetrahydrofuran.

The term “heterocycle”used alone or as a suffix or prefix, refers to a ring structure or molecule of at least three and up to 20 atoms having one or more multivalent heteroatoms, such atoms independently selected from O, N, P or S as part of the ring structure. Heterocycles may be saturated, partially-saturated or unsaturated, may have atoms linked by on or more double bonds and may form one or more rings that may be linked or fused, where fused rings share at least two atoms therebetween. Heterocycles may or may not have aromatic character.

Temperatures are given in degrees Celsius (° C.); unless otherwise stated, operations were carried out at room or ambient temperature (18-25° C.).

Organic solutions were dried over anhydrous sodium or magnesium sulfate; evaporation of solvent was carried out using a rotary evaporator under reduced pressure (4.5-30 mm Hg) with a bath temperature of up to 60° C.

Chromatography means flash column chromatography on silica gel unless otherwise noted; solvent mixture compositions are given as volume percentages or volume ratios.

When given, NMR data is in the form of delta values for major diagnostic protons (given in parts per million (ppm) relative to tetramethylsilane as an internal standard) determined at 300 MHz.

Melting points are uncorrected.

Mass spectra (MS) were obtained using an automated system with atm chemical ionization (APCI) unless otherwise indicated. Masses corresponding to the major isotopic component, or the lowest mass for compounds with multiple masses with nearly equivalent abundance (isotope splitting), are reported.

Where noted that a final compound was converted to the citrate salt, the free base was dissolved in MeOH, DCM, or ACN, combined with citric acid (1.0 equivalents) in MeOH, concentrated under reduced pressure and dried under vacuum (25-60° C.). When indicated that the salt was isolated by filtration from Et₂O, the citrate salt of the compound was stirred in Et₂O for 4-18 h, recovered by filtration, washed with Et₂O, and dried under vacuum (25-60° C.).

### Example 1

### N-[(3-Cyano-2-methoxy-1-naphthyl)methyl]-2-(3-methoxyphenyl)-N-methyl-2-(4-methylpiperazin-1-yl)acetamide

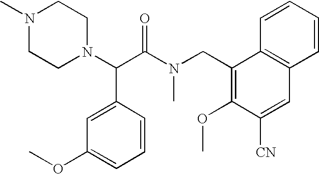

N-[(3-cyano-2-methoxy-1-naphthyl)methyl]-2-(3-methoxyphenyl)-N-methyl-2-piperazin-1-ylacetamide (40 mg, 0.087 mmol), formic acid (0.25 mL, 6.4 mmol) and formaldehyde (37% aq., 1.9 mL, 26 mmol) were reacted for 1 h at 100° C. The cooled reaction was neutralized with sat. aq. NaHCO₃ and extracted with DCM. The organic phase was dried over Na₂SO₄, filtered through a pad of diatomaceous earth and the volatiles removed under reduce pressure. Chromatography of the residue on SiO₂ (0-5% MeOH:DCM) afforded the title compound (27 mg, 66%). MS m/z 473.3 (M+H)⁺. ¹H NMR (300.1 MHz, DMSO) δ 8.60 (s, 1H), 8.01 (d, J=8.0 Hz, 1H), 7.94 (d, J=8.0 Hz, 1H), 7.60-7.50 (m, 2H), 7.20 (t, J=8.0 Hz, 1H), 6.98-6.96 (m, 2H), 6.83 (d, J=8.0 Hz, 1H), 5.03 (dd, J=19.5, 14.3 Hz, 2H), 4.40 (s, 1H), 3.94 (s, 3H), 3.94 (s, 3H), 3.69 (s, 3H), 2.44 (broad s, 4H), 2.44 (broad s, 4H), 2.14 (s, 3H). The citrate salt was formed by the addition of citric acid (11 mg, 1.0 equivalents) to a methanolic solution of the title compound (27 mg). Concentration under reduced pressure afforded the desired salt form of the product as a white foam. MS m/z 473.3 (M+H)⁺.

The requisite N-[(3-cyano-2-methoxy-1-naphthyl)methyl]-2-(3-methoxyphenyl)-N-methyl-2-piperazin-1-ylacetamide was synthesized using the following method.

### Example 2

### N-[(3-cyano-2-methoxy-1-naphthyl)methyl]-2-(3-methoxyphenyl)-N-methyl-2-piperazin-1-ylacetamide

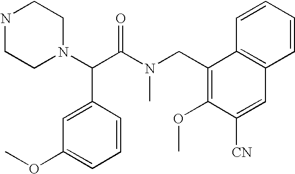

tert-Butyl 4-[2-[[(3-cyano-2-methoxy-1-naphthyl)methyl](methyl)amino]-1-(3-methoxyphenyl)-2-oxoethyl]piperazine-1-carboxylate (123 mg, 0.22 mmol) was deprotected in 1:1 TFA:DCM (20 mL). After 1 h, the volatiles were removed under reduced pressure. The residue was dissolved in DCM, washed with sat. aq. NaHCO₃. The organic phase was dried over Na₂SO₄, filtered through a pad of diatomaceous earth and the volatiles removed under reduce pressure. Chromatography of the residue on SiO₂ (0-5% 2 M NH₃ in MeOH:DCM) afforded the title compound (83 mg, 82%). MS m/z 459.2 (M+H)⁺. ¹H NMR (300.1 MHz, DMSO) δ 8.60 (s, 1H), 8.01 (d, J=7.4 Hz, 1H), 7.96 (d, J=8.2 Hz, 1H), 7.61-7.52 (m, 2H), 7.20 (t, J=8.2 Hz, 1H), 6.97 (s, 2H), 6.83 (d, J=8.2 Hz, 1H), 5.03 (dd, J=21.3, 14.2 Hz, 2H), 4.43 (s, 1H), 3.95 (s, 3H), 3.69 (s, 3H), 2.71-2.69 (m, 7H), 2.43-2.41 (m, 4H). The citrate salt was formed by the addition of citric acid (16 mg, 1.0 equivalents) to a methanolic solution of the title compound (39 mg). Concentration under reduced pressure afforded the desired salt form of the product as a white foam. MS m/z 459.3 (M+H)⁺.

The requisite tert-butyl 4-[2-[[(3-cyano-2-methoxy-1-naphthyl)methyl](methyl)amino]-1-(3-methoxyphenyl)-2-oxoethyl]piperazine-1-carboxylate was synthesized using the following method.

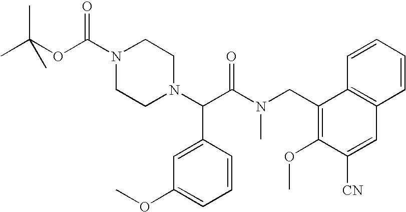

[4-(tert-Butoxycarbonyl)piperazin-1-yl](3-methoxyphenyl)acetic acid (100 mg, 0.28 mmol), 3-methoxy-4-[(methylamino)methyl]-2-naphthonitrile (64 mg, 0.28 mmol), HOBT (58 mg, 0.43 mmol), and 1-(3-dimethylaminopropyl)-3-ethylcarbodiimide hydrochloride (66 mg, 0.34 mmol) were reacted together in DCM (10 mL) at RT overnight. The reaction mixture was partitioned between water (20 mL) and DCM (20 mL). The organic layer was washed with sat. aq. NaHCO₃, dried over Na₂SO₄, filtered through a pad of diatomaceous earth and the volatiles were removed under reduced pressure. Chromatography of the residue on SiO₂ (0-50% EtOAc:hexane) afforded the title compound as a white solid (123 mg, 77%). MS m/z 559.4 (M+H)⁺. ¹H NMR (300.1 MHz, DMSO) δ 8.60 (s, 1H), 8.04-7.96 (m, 2H), 7.61-7.54 (m, 2H), 7.21 (t, J=8.3 Hz, 1H), 6.97-6.95 (m, 2H), 6.84 (dd, J=8.3, 2.1 Hz, 1H), 5.04 (s, 2H), 4.51 (s, 1H), 3.95 (s, 3H), 3.69 (s, 3H), 3.29-3.26 (m, 4H), 2.67 (s, 3H), 2.47-2.37 (m, 4H), 1.38 (s, 9H).

The requisite [4-(tert-butoxycarbonyl)piperazin-1-yl](3-methoxyphenyl)acetic acid was synthesized using the following method.

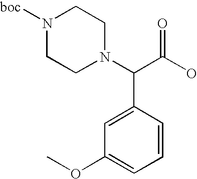

(3-Methoxyphenyl)boronic acid (345 mg, 1.61 mmol), tert-butyl piperazine-1-carboxylate (300 mg, 1.61 mmol) and glyoxylic acid monohydrate (148 mg, 1.61 mmol) were reacted together in DCM (10 mL) at reflux overnight. The volatiles were removed under reduced pressure. Chromatography of the residue on SiO₂ (0-10% MeOH:DCM) afforded the title compound (531 mg, 94%). MS m/z 351.1 (M+H)⁺, 295.1 (M+H-t-butyl). ¹H NMR (300.1 MHz, DMSO) δ 12.39 (s, 0.2H), 7.27 (t, J=8.4 Hz, 1H), 6.97-6.94 (m, 2H), 6.89 (d, J=8.4 Hz, 1H), 3.97 (s, 1H), 3.74 (s, 3H), 3.36-3.26 (m, 10H [4H +H₂O]), 2.40-2.31 (m, 4H), 1.37 (s, 9H).

### Example 3

### N-[(3-cyano-2-methoxy-1-naphthyl)methyl]-2-(4-fluorophenyl)-N-methyl-2-pyrrolidin-1-ylacetamide

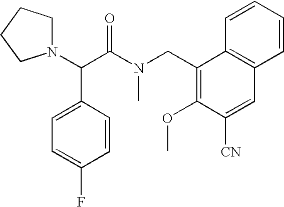

(4-Fluorophenyl)(pyrrolidin-1-yl)acetic acid (400 mg, 1.79 mmol), 3-methoxy-4-[(methylamino)methyl]-2-naphthonitrile (405 mg, 1.79 mmol), HOBT (363 mg, 2.69 mmol), and 1-(3-dimethylaminopropyl)-3-ethylcarbodiimide hydrochloride (412 mg, 2.15 mmol) were reacted together in DCM (15 mL) at RT overnight. The reaction mixture was partitioned between water (20 mL) and DCM (20 mL). The organic layer was washed with sat. aq. NaHCO₃, dried over Na₂SO₄, filtered through a pad of diatomaceous earth and the volatiles were removed under reduced pressure. Chromatography of the residue on SiO₂ (35 g) (0-5% MeOH:DCM) afforded the title compound as a white solid (300 mg). ¹H NMR (300.1 MHz, DMSO) δ 8.61 (s, 6H), 8.04 (d, J=8.5 Hz, 7H), 7.91 (d, J=8.5 Hz, 7H), 7.71-7.58 (m, 26H), 7.29 (t, J=8.5 Hz, 12H), 5.70 (s, 6H), 5.24 (d, J=14.3 Hz, 6H), 5.00 (d, J=14.3 Hz, 6H), 3.98 (s, 18H), 3.78-3.72 (m, 7H), 3.23-3.14 (m, 6H), 2.99-2.90 (m, 7H), 2.82-2.76 (m, 7H), 2.61 (s, 19H), 2.06-1.96 (m, 19H), 1.90-1.78 (m, 8H). The citrate salt was formed by the addition of citric acid (1.0 equivalents) to a methanolic solution of the title compound. Concentration under reduced pressure afforded the desired salt form of the product as a white foam. MS m/z 432.2 (M+H)⁺.

The requisite (4-fluorophenyl)(pyrrolidin-1-yl)acetic acid was synthesized using the following method.

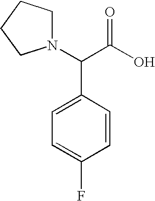

Pyrrolidine (200 mg, 2.8 mmol), glyoxylic acid monohydrate (258 mg, 2.8 mmol) and (4-fluorophenyl)boronic acid (393 mg, 2.8 mmol) were reacted together in DCM (10 mL) at reflux overnight. The volatiles were removed under reduced pressure, the resulting residue was dissolved in DCM (1 mL). The solution was added to vigorously stirred Et₂O (100 mL) to afford the product as a pale orange powder (430 mg). MS m/z 224.1 (M+H)⁺.

The requisite 3-methoxy-4-[(methylamino)methyl]-2-naphthonitrile was synthesized using the following method.

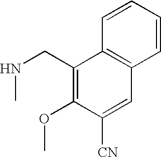

Methylamine (500 mL of a 2.0 M solution in MeOH) was added to 4-(iodomethyl)-3-methoxy-2-naphthonitrile (5.00 g, 15.5 mmol). After reacting at RT overnight in a sealed vessel, the volatiles were removed under reduced pressure. The residue was taken up in DCM (350 mL) and washed with sat. aq. NaHCO₃ (550 mL). The organic layer was dried over Na₂SO₄, filtered through though a pad of diatomaceous earth and the volatiles were removed under reduced pressure to afford the title compound as a pale yellow powder (3.50 g, 100%). ¹H NMR (300.1 MHz, DMSO) δ 8.53 (s, 1H), 8.26 (d, J=8.4 Hz, 1H), 8.01 (d, J=8.4 Hz, 1H), 7.72 (ddd, J=8.4, 7.0, 1.4 Hz, 1H), 7.62-7.56 (m, 1H), 4.06 (s, 2H), 3.94 (s, 3H), 3.31 (s, 1H), 2.38 (s, 3H).

### Example 4

### Chiral Separation of N-[(3-cyano-2-methoxy-1-naphthyl)methyl]-2-(4-fluorophenyl)-N-methyl-2-pyrrolidin-1-ylacetamide

Racemic mixture of N-[(3-cyano-2-methoxy-1-naphthyl)methyl]-2-(4-fluorophenyl)-N-methyl-2-pyrrolidin-1-ylacetamide was separated into its component enantiomers using preparative supercritical fluid chromatography on a Chiralpak AD-H column (20×250 mm, 5 μm) with an eluent consisting of 20% methanol containing 0.5% dimethylethylamine and carbon dioxide at a flow rate of 50 mL/min with detection at 280 nm. Chiral purity was assessed by analysis with supercritical fluid chromatography on a Chiralpak AD-H column (4.6×250 mm, 5 μm) with an eluent consisting of 20% methanol containing 0.5% dimethylethylamine and carbon dioxide at a flow rate of 2.2 mL/min with detection at 280 nm. Isomer 1: T_{R}=3.54 min; >99% ee. Isomer 2: T_{R}=4.32 min; >99% ee.

### Example 5

### N-[(3-cyano-2-methoxy-1-naphthyl)methyl]-2-(4-fluorophenyl)-N-methyl-2-[4-(trifluoroacetyl)piperazin-1-yl]acetamide

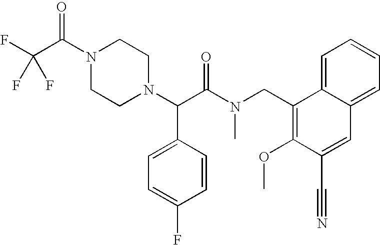

Trifluoroacetic anhydride (0.035 mL, 0.25 mmol) was added to a solution of N-[(3-cyano-2-methoxy-1-naphthyl)methyl]-2-(4-fluorophenyl)-N-methyl-2-piperazin 1-ylacetamide (0.10 g, 0.22 mmol) and diisopropylethylamine (0.078 mL, 0.45 mmol) in DCM (10 mL). After 2 h the reaction was quenched with water (10 mL) for 15 min. The organic layer was washed with 1N HCl (10 mL), sat. aq. NaHCO₃ (10 mL) then H₂O (10 mL). The organic layer was dried over Na₂SO₄, filtered through diatomaceous earth and the volatiles were removed under reduced pressure. Chromatography of the residue on silica gel (4 g; 0-5% MeOH:DCM) afforded the title compound (0.065 g, 54%). ¹H NMR (300.132 MHz, DMSO) δ 8.60 (s, 1H), 8.02 (dd, J=6.2, 3.4 Hz, 1H), 7.93 (dd, J=6.0, 3.3 Hz, 1H), 7.59 (dd, J=6.4, 3.2 Hz, 2H), 7.46 (dd, J=8.5, 5.6 Hz, 2H), 7.14 (t, J=8.8 Hz, 2H), 5.10-5.00 (m, 2H), 4.68 (s, 1H), 3.96 (s, 3H), 3.55-3.53 (m, 4H), 2.66 (s, 3H), 2.61-2.56 (m, 4H). The citrate salt was formed by the addition of citric acid (1.0 equivalents) to a methanolic solution of the title compound. Concentration under reduced pressure afforded the desired salt form of the product as a solid. MS m/z 543.3 (+H)⁺.

The requisite N-[(3-cyano-2-methoxy-1-naphthyl)methyl]-2-(4-fluorophenyl)-N-methyl-2-piperazin-1-ylacetamide was synthesized in a manner analogous to that described in Example 2.

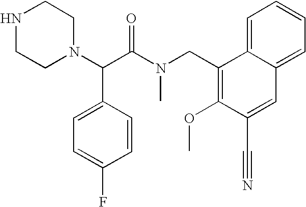

### Example 6

### N-[(3-cyano-2-methoxy-1-naphthyl)methyl]-2-(4-ethylpiperazin-1-yl)-2-(4-fluorophenyl)-N-methylacetamide

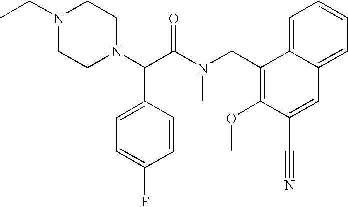

N-[(3-cyano-2-methoxy-1-naphthyl)methyl]-2-(4-fluorophenyl)-N-methyl-2-piperazin-1-ylacetamide (0.10 g, 0.22 mmol) was added to a solution of acetaldehyde (0.012 mL, 0.20 mmol) in THF (5 mL). After 5 min, macroporous triethylammonium methylpolystyrene triacetoxyborohydride (0.21 g, 2.07 mmol/g) was added. After 18 h the reaction was filtered through a plug of diatomaceous earth and the filtrate was concentrated under reduced pressure. The residue was purified using silica gel chromatography (4 g; 0-10% [2M NH₃ in MeOH]:DCM) to afford the title compound (0.051 g, 53%). MS m/z 575.3 (M+H)⁺. ¹H NMR (300.132 MHz, DMSO) δ 8.59 (s, 1H), 8.01 (d, J=7.5 Hz, 1H), 7.89 (d, J =8.4 Hz, 1H), 7.60-7.51 (m, 2H), 7.49-7.44 (m, 2H), 7.12 (t, J=8.8 Hz, 2H), 5.03 (dd, J=18.5, 14.3 Hz, 2H), 4.48 (s, 1H), 3.95 (s, 3H), 3.29-3.27 (m, 2H), 2.71 (s, 3H), 2.43-2.31 (m, 8H), 0.98-0.94 (m, 3H). The citrate salt was formed by the addition of citric acid (1.0 equivalents) to a methanolic solution of the title compound. Concentration under reduced pressure afforded the desired salt form of the product as a solid.

### Example 7

### N-[(3-cyano-2-methoxy-1-naphthyl)methyl]-2-(4-fluorophenyl)-N-methyl-2-[4-(2,2,2-trifluoroethyl)piperazin-1-yl]acetamide

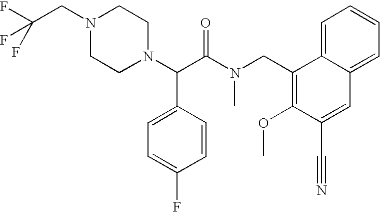

2,2,2-Trifluoroethyl trifluoromethanesulfonate (0.052 g, 0.22 mmol) was added to a solution of N-[(3-cyano-2-methoxy-1-naphthyl)methyl]-2-(4-fluorophenyl)-N-methyl-2-piperazin-1-ylacetamide (0.10 g, 0.22 mmol) and diisopropylethylamine (0.078 mL, 0.45 mmol) in benzene (10 mL). After the reaction was refluxed for 18 h, it was cooled to room temperature, quenched with H₂O (10 mL) and diluted with DCM (25 mL). The organic layer was washed with sat. aq. NaHCO₃ (15 mL) then with sat. aq. NaCl (10 mL), dried over Na₂SO₄, filtered through diatomaceous earth and concentrated under reduced pressure. The residue was purified by silica gel chromatography (4 g; 0-5% MeOH:DCM) to afford the title compound (0.089 g, 75%). MS m/z 529.2 (M+H)⁺. ¹H NMR (300.132 MHz, DMSO) δ 8.60 (s, 1H), 8.01 (d, J=7.3 Hz, 1H), 7.90 (d, J=7.3 Hz, 1H), 7.60-7.52 (m, 2H), 7.46 (dd, J=8.4, 5.9 Hz, 2H), 7.12 (t, J=8.8 Hz, 2H), 5.04 (dd, J=16.4, 14.3 Hz, 2H), 4.52 (s, 1H), 3.95 (s, 3H), 3.10 (q, J=10.2 Hz, 2H), 2.71 (s, 3H), 2.56 (br s, 4H), 2.46-2.43 (m, 4H). The citrate salt was formed by the addition of citric acid (1.0 equivalents) to a methanolic solution of the title compound. Concentration under reduced pressure afforded the desired salt form of the product as a solid.

### Example 8

### 2-(allylamino)-N-[(3-cyano-2-methoxy-1-naphthyl)methyl]-2-(3-methoxyphenyl)-N-methylacetamide

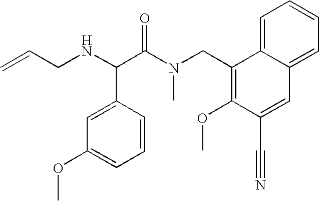

Bis(dibenzylideneacetone)palladium (0) (0.049 g, 0.0852 mmol) and 1,4-bis(diphenylphosphino)butane (0.036 g, 0.0852 mmol) were added to THF (2 mL) that had been purged with N₂ for 5 min. After 20 min, the mixture was added to a solution of N-[(3-cyano-2-methoxy-1-naphthyl)methyl]-2-(diallylamino)-2-(3-methoxyphenyl)-N-methylacetamide (0.200 g, 0.426 mmol) in THF (10 mL). A solution of 2-mercaptobenzoic acid (0.144 g, 0.937 mmol) in THF (3 mL) was added drop wise. The reaction was heated to 80° C. for 18 h. The reaction was cooled to room temperature, diluted with EtOAc and the organic layer was washed with sat. aq. NaHCO₃, H₂O and sat. aq. NaCl. The organic layer was dried over Na₂SO₄, filtered through diatomaceous earth and concentrated. The residue was purified using silica gel chromatography (12 g, 0-5% MeOH:DCM) to afford the title compound (0.012 g, 7%) as well as the fully deprotected amine 2-amino-N-[(3-cyano-2-methoxy-1-naphthyl)methyl]-2-(3-methoxyphenyl)-N-methylacetamide (0.070 g, 42%). 2-(Allylamino)-N-[(3-cyano-2-methoxy-1-naphthyl)methyl]-2-(3-methoxyphenyl)-N-methylacetamide: MS m/z 430.2 (M+H)⁺. The citrate salt was formed by the addition of citric acid (1.0 equivalents) to a methanolic solution of the title compound. Concentration under reduced pressure afforded the desired salt form of the product as a solid. ¹H NMR (300.132 MHz, DMSO) δ 8.61 (s, 1H), 8.01 (br s, 2H), 7.62-7.60 (m, 2H), 7.28-7.18 (m, 1H), 6.99 (br s, 2H), 6.89 (d, J=8.0 Hz, 1H), 5.92-5.83 (m, 1H), 5.28-5.18 (m, 4H), 5.13-5.08 (m, 2H), 4.98 (d, J=14.3 Hz, 1H), 3.96 (s, 3H), 3.70 (s, 3H), 2.63 (dd, J=29.5, 15.1 Hz, 4H), 2.61 (s, 3H). 2-Amino-N-[(3-cyano-2-methoxy-1-naphthyl)methyl]-2-(3-methoxyphenyl)-N-methylacetamide: MS m/z 390.1 (M+H)⁺. ¹H NMR (300.132 MHz, DMSO) δ 8.60 (s, 1H), 8.04-8.00 (m, 2H), 7.63-7.56 (m, 2H), 7.15 (t, J=8.0 Hz, 1H), 6.88 (d, J=7.7 Hz, 1H), 6.87 (s, 1H), 6.76 (d, J=9.2 Hz, 1H), 5.26 (d, J=14.0 Hz, 1H), 4.87 (d, J=14.2 Hz, 1H), 4.84 (s, 1H), 3.96 (s, 3H), 3.64 (s, 3H), 3.31 (br s, 2H), 2.61 (s, 3H).

### Example 9

### N-[(3-cyano-2-methoxy-1-naphthyl)methyl]-2-(4,4-difluoropiperidin-1-yl-2-(3-methoxyphenyl)-N-methylacetamide

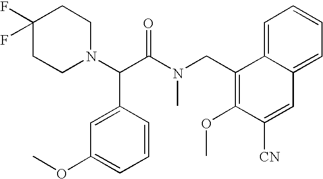

(4,4-Difluoropiperidin-1-yl)(3-methoxyphenyl)acetic acid (0.064 g, 0.22 mmol), 3-methoxy-4-[(methylamino)methyl]-2-naphthonitrile (0.053 g, 0.24 mmol), 1-(3-dimethylaminopropyl)-3-ethylcarbodiimide hydrochloride (0.129 g, 0.66 mmol), HOBT (0.045 g, 0.33 mmol), and 4-dimethylaminopyridine (0.027 g, 0.22 mmol) were reacted together in DCM (0.9 mL). After 18 h the reaction was diluted with DCM (15 mL) and washed with H₂O (10 mL) and sat. aq. NaHCO₃ (10 mL). The organic layer was dried over Na₂SO₄, filtered through diatomaceous earth and concentrated under reduced pressure. Chromatography of the residue on SiO₂ (4 g; 0-8% [2M NH₃ in MeOH]:DCM) afforded the title compound as a white solid (0.062 g, 56%). The citrate salt was formed by the addition of citric acid (1.0 equivalents) to a methanolic solution of the title compound. Concentration under reduced pressure afforded the desired salt form of the product as a white foam. MS m/z 494.2 (M+H)⁺. ¹H NMR (300.132 MHz, MeOH) δ 8.36 (s, 1H), 8.06 (d, J=8.4 Hz, 1H), 7.94 (d, J=7.7 Hz, 1H), 7.64 (t, J=7.2 Hz, 1 H), 7.55 (t, J=7.3 Hz, 1H), 7.35 (t, J=7.7 Hz, 1H), 7.12 (s, 1H), 7.11 (d, J=8.0 Hz, 1H), 7.02 (d, J=7.9 Hz, 1H), 5.49 (t, J=6.9 Hz, 2H), 5.00 (d, J=14.3 Hz, 1H), 4.04 (s, 3H), 3.77 (s, 3H), 3.37-3.24 (m, 4H), 2.87 (dd, J=37.4, 15.7 Hz, 4H), 2.67 (s, 3H), 2.42 (s, 4H).

The requisite (4,4-difluoropiperidin-1-yl)(3-methoxyphenyl)acetic acid was synthesized using the following method.

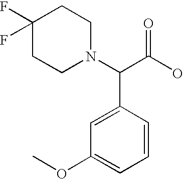

4,4-Difluoropiperidine hydrochloride (473 mg, 3.00 mmol) and diisopropylethylamine (0.026 mL, 0.015 mmol) were mixed with macroporous triethylammonium methylpolystyrene carbonate resin (3.75 g, 3.2 mmol/g) in DCM (10 mL) for 2 h. The resin was removed by filtration. The resin was rinsed with DCM (2×5 mL). The flow through and washings were collected and combined. One half of this 4,4-difluoropiperidine solution in DCM was added to a mixture of (3-methoxyphenyl)boronic acid (228 mg, 1.5 mmol) and glyoxylic acid monohydrate (138 mg, 1.5 mmol). The reaction was heated to reflux for 16 h. The reaction was cooled and the volatiles were removed under reduced pressure. The resulting residue was dissolved in EtOAc (15 mL) and extracted with H₂O (2×15 mL). The combined aqueous layers were filtered through a filter paper then concentrated to dryness affording the title compound as a white solid (288 mg, 67%). MS m/z 286.2 (M+H)⁺. ¹H NMR (300.132 MHz, DMSO) δ 7.48 (dd, J=9.1, 7.3 Hz, 1H), 7.14-7.11 (m, 3H), 5.38 (s, 1H), 3.82 (s, 3H), 3.58 (s, 2H), 3.14 (dt, J=17.0, 7.3 Hz, 2H), 2.37 (s, 4H).

### Example 10

### N-[(3-cyano-2-ethyl-1-naphthyl)methyl]-2-(4-fluorophenyl)-N-methyl-2-morpholin-4-ylacetamide

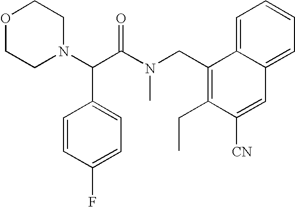

(4-Fluorophenyl)(morpholin-4-yl)acetic acid (0.112 g, 0.47 mmol), 3-ethyl-4-[(methylamino)methyl]-2-naphthonitrile (0.110 g, 0.49 mmol), 1-(3-dimethylaminopropyl)-3-ethylcarbodiimide hydrochloride (0.268 g, 1.41 mmol), HOBT (0.095 g, 0.71 mmol), and 4-dimethylaminopyridine (0.057 g, 0.47 mmol) were reacted together in DCM (1.9 mL). After 24 h the reaction was diluted with DCM (10 mL) and washed with H₂O (10 mL) and sat. aq. NaHCO₃ (10 mL). The organic layer was dried over Na₂SO₄, filtered through diatomaceous earth and concentrated under reduced pressure. Chromatography of the residue on SiO₂ (4 g; 0-5% [2M NH₃ in MeOH]:DCM) afforded the title compound (0.083 g, 40%). MS m/z 446.3 (M+H)⁺. ¹H NMR (300.132 MHz, DMSO) δ 8.56 (s, 1H), 8.00 (d, J=7.8 Hz, 1H), 7.91 (d, J=8.2 Hz, 1H), 7.61-7.48 (m, 4H), 7.17 (t, J=8.8 Hz, 2H), 5.10 (dd, J=23.9, 14.8 Hz, 2H), 4.52 (s, 1H), 3.57-3.54 (m, 4H), 3.03 (q, J=7.3 Hz, 2H), 2.64 (s, 3H), 2.48-2.39 (m, 4H), 1.16 (t, J=7.4 Hz, 3H). The citrate salt was formed by the addition of citric acid (1.0 equivalents) to a methanolic solution of the title compound. Concentration under reduced pressure afforded the desired salt form of the product as a white solid.

The requisite 3-ethyl-4-[(methylamino)methyl]-2-naphthonitrile was synthesized using the following method.

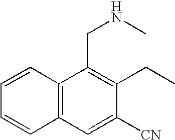

To the round bottom flask containing 3-ethyl-4-(iodomethyl)-2-naphthonitrile (3.51 g, 10.9 mmol) was poured methylamine (2M in MeOH, 100 mL). The reaction mixture was stirred at room temperature over night. Solvent was evaporated and sat. aq. NaHCO₃ was added. The mixture was extracted with DCM, dried over MgSO₄, filtered and concentrated. Chromatography of the residue on SiO₂ (0-5% MeOH:DCM) afforded the title compound (2.16 g, 89%). ¹H NMR (300.1 MHz, CDCl₃) δ 8.17 (t, 2H), 7.83 (d, 1H), 7.66 (t, 1H), 7.47 (t, 1H), 4.17 (s, 2H), 3.12 (q, 2H), 2.62 (s, 3H), 1.35 (t, 3H).

The requisite (4-fluorophenyl)(morpholin-4-yl)acetic acid was synthesized in manner analogous to that described in Example 2.

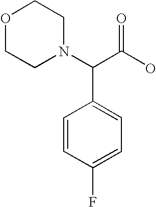

### Example 11

### N-[(3-bromo-2-methoxy-5,6,7,8-tetrahydronaphthalen-1-yl)methyl]-2-(4-fluorophenyl)-N-methyl-2-piperazin-1-ylacetamide

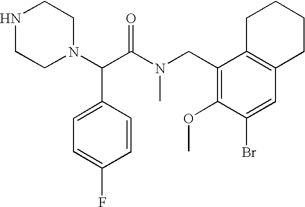

tert-Butyl 4-[2-[[(3-bromo-2-methoxy-5,6,7,8-tetrahydronaphthalen-1-yl)methyl](methyl)amino]-1-(4-fluorophenyl)-2-oxoethyl]piperazine-1-carboxylate (130 mg, 0.22 mmol) was deprotected in 1:1 TFA:DCM (20 mL). After 4 h, the volatiles were removed under reduced pressure. The residue was dissolved in DCM (30 mL) and washed with sat. aq. NaHCO₃ (50 mL). The organic phase was dried over Na₂SO₄, filtered through a pad of diatomaceous earth and the volatiles removed under reduce pressure. Chromatography of the residue on SiO₂ (4 g; 0-10% 2 M NH₃ in MeOH:DCM) afforded the title compound (84 mg). MS m/z 504.3, 506.0 (M+H)⁺. ¹H NMR (300.132 MHz, DMSO) δ 7.63 (dd, J=8.6, 5.3 Hz, 2H), 7.41 (t, J=8.8 Hz, 2H), 7.29 (s, 1H), 5.91 (s, 1H), 4.94 (d, J=14.2 Hz, 1H), 4.47 (d, J=14.2 Hz, 11H), 3.70 (s, 3H), 3.50-3.39 (m, 6H), 3.24 (br s, 2H), 2.75 (dd, J=33.0, 15.4 Hz, 4H), 2.64 (br s, 2H), 2.61 (s, 3H), 2.44-2.38 (m, 1H), 2.01-1.96 (m, 1H), 1.62-1.50 (m, 4H). The citrate salt was formed by the addition of citric acid (22 mg, 1.0 equivalents) to a methanolic solution of the title compound (59 mg). Concentration under reduced pressure afforded the desired salt form of the product as a white solid.

The requisite tert-butyl 4-[2-[[(3-bromo-2-methoxy-5,6,7,8-tetrahydronaphthalen-1-yl)methyl](methyl)amino]-1-(4-fluorophenyl)-2-oxoethyl]piperazine-1-carboxylate was synthesized using the following method.

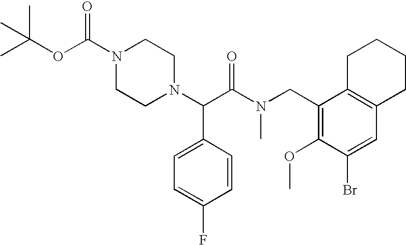

[(3-Bromo-2-methoxy-5,6,7,8-tetrahydronaphthalen-1-yl)methyl]methylamine (0.35 g, 1.23 mmol), 1-ethyl-3-(3-dimethylaminopropyl)carbodiimide hydrochloride (0.47 g, 2.46 mmol), 1-hydroxybenzotriazole (0.17 g, 1.23 mmol), triethylamine (0.69 mL, 4.92 mmol) and [4-(tert-butoxycarbonyl)piperazin-1-yl](4-fluorophenyl)acetic acid (0.50 g, 1.48 mmol) were combined in DCM (50 mL) and stirred at room temperature for 12 h. The solution was diluted with EtOAc (200 mL) and washed with 1N NaOH (2×50 mL). The organics were dried over Na₂SO₄, filtered and the volatiles removed under reduced pressure. The residue was purified utilizing silica gel chromatography (40 g; 0-10% 2M (NH₃/MeOH):DCM. The product was isolated as a clear oil (130 mg, 17% yield). MS m/z 606.3 (M+H)⁺. ¹H NMR (300.132 MHz, CD₃OD at 65° C.) δ 0.90 (m, 1H), 1.09 (q, J=2.5 Hz, 1H), 1.25 (t, J=3.8 Hz, 2H), 1.30 (s, 4H), 1.41 (s, 9H), 1.68 (m, 4H), 1.99 (s, 1H), 2.15 (m, 2H), 2.42 (m, 3H), 2.71 (m, 4H), 3.32 (s, 2H), 3.42, (br s, 4H), 3.75 (m, 4H), 4.13 (m, 5H), 4.44 (m, 1H), 4.75 (m, 2H), 7.06 (t, J=4.2 Hz, 2H), 7.23 (s, 1H), 7.46 (m, 2H).

The requisite [(3-bromo-2-methoxy-5,6,7,8-tetrahydronaphthalen-1-yl)methyl]-methylamine was synthesized using the following method.

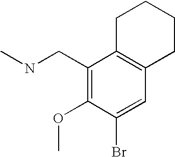

3-Bromo-1-(bromomethyl)-5,6,7,8-tetrahydronapththalen-2-yl methyl ether (0.60 g, 1.80 mmol) was dissolved in 20 mL of anhydrous ethanol. A 33% methyl amine/ethanol solution (100 mL) was added to the flask and the reaction allowed to stir overnight at room temperature. The volatiles were removed and the product used without further purification. MS m/z 284.9 (M+H)⁺. ¹H NMR (300.1 MHz, CDCl₃) δ 1.65. (br s, 4H), 2.71-2.53 (m, 7H), 3.82 (s, 3H), 4.02 (s, 3H), 7.17 (s, 1H).

The requisite 3-bromo-1-(bromomethyl)-5,6,7,8-tetrahydronapththalen-2-yl methyl ether was synthesized using the following method.

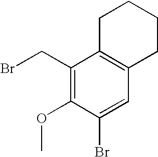

Carbon tetrabromide (3.82 g, 11.2 mmol) and triphenylphosphine (3.02 g, 11.5 mmol) were added to a solution of (3-bromo-2-methoxy-5,6,7,8-tetrahydronaphthalen-1-yl)methanol (2.84 g, 10.5 mmol) in anhydrous DCM (100 mL). The reaction was allowed to stir for 48 h at room temperature. The solution was filtered through a plug of silica gel and the product eluted with diethyl ether. The volatiles were removed under reduced pressure and the title compound was obtained as a clear oil (3.36 g, 96%). ¹H NMR (300.1 MHz, CDCl₃) δ 1.75-1.90. (m, 4H), 2.71-2.84 (m, 4H), 3.97 (s, 3H), 4.73 (s, 2H), 7.29 (s, 1H).

The requisite (3-bromo-2-methoxy-5,6,7,8-tetrahydronaphthalen-1-yl)methanol was synthesized using the following method.

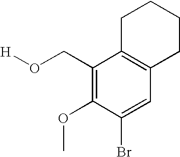

3-Bromo-2-methoxy-5,6,7-8-tetrahydronaphthalene-1-carboxylic acid (6.00 g, 21.0 mmol) was dissolved in 100 mL of anhydrous THF. Borane THF complex (20.0 mL, 210 mmol) was added and the reaction was allowed to proceed at room temperature over 72 h. The reaction was cooled in an ice-water bath and the reaction quenched with the drop wise addition of methanol (25 mL) over a 2 h period. The volatiles were removed under reduced pressure and the product purified on silica gel (120g; 0-70% EtOAc:hexane). The product was obtained as a clear oil that solidified upon prolonged standing (2.84 g, 50%). MS m/z 253.1 (M−OH)⁺. ¹H NMR (300.1 MHz, CDCl₃) δ 1.71-1.86 (m, 4H), 2.04 (s, 1H), 2.73 (t, J=6.1 Hz, 1H), 2.82 (t, J=6.1 Hz, 1H), 3.88 (s, 3H), 4.75, (s, 2H), 7.26 (s, 1H).

The requisite 3-bromo-2-methoxy-5,6,7-8-tetrahydronaphthalene-1-carboxylic acid was synthesized using the following method.

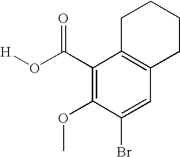

Methyl 3-bromo-2-methoxy-5,6,7,8-tetrahydronaphthalene-1-carboxylate (8.3 g, 27.7 mmol) was dissolved in a solution of MeOH/THF/H₂O (1:1:1; 450 mL) followed by the addition of KOH (15.56 g, 277.4 mmol) and the solution heated to a gentle reflux overnight. The reaction was cooled to room temperature and diluted with diethyl ether (400 mL). The product was extracted with H₂O (2×200 mL). The aqueous extract was acidified to pH 1 with 1 N HCl and the final product extracted with EtOAc (2×200 mL). The organic layer was dried over MgSO₄, filtered and the volatiles removed under reduced pressure to give the title product as a brownish oil (5.87 g, 74%). ¹H NMR (300.132 MHz, CDCl₃) δ 1.77 s, 4H), 2.72-2.77 (br s, 4H), 3.93 (s, 3H), 7.35 (s, 1H), 11.75 (br s, 1H).

### Example 12

### N-[(3-cyano-2-methoxy-1-naphthyl)methyl]-2-[(3′S)-2,5-dioxo-1,3′-bipyrrolidin-1′-yl]-2-(4-fluorophenyl)-N-methylacetamide

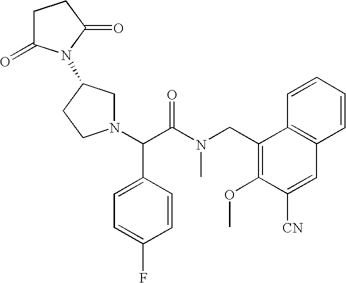

To the flask containing 2-[(3S)-3-aminopyrrolidin-1-yl]-N-[(3-cyano-2-methoxy-1-naphthyl)methyl]-2-(4-fluorophenyl)-N-methylacetamide (120 mg, 0.269 mmol) in dioxane (2.5 mL) was added succinic anhydride (27 mg, 0.269 mmol). The reaction mixture was refluxed for 1 h. After cooling to room temperature, triethylamine (0.075 mL, 0.538 mmol) was added. The mixture was refluxed for over night. Solvent was evaporated. Chromatography of the residue on SiO₂ (0-1.5% MeOH:DCM) afforded the title compound (48 mg, 34%). MS m/z 529.3 (M+H)⁺. ¹H NMR (300.1 MHz, CDCl₃) δ 8.17 (s, 1H), 8.07 (m, 1H), 7.81 (m, 1H), 77.5-7.40 (m, 4H), 6.96 (m, 2H), 5.24-5.08 (m), 4.89-4.77 (m), 4.37-4.24 (m), 4.00 (s, 3H), 3.32 (m, 1H), 2.74-2.61 (m), 2.47-2.05 (m). The citrate salt was formed by the addition of citric acid (17 mg, 1.0 equivalent) to a methanolic solution of the title compound (48 mg). Concentration under reduced pressure afforded the desired salt form of the product as a white foam.

### Example 13

### N-{(3S-1-[2-[[(3-cyano-2-methoxy-1-naphthyl)methyl](methyl)amino]1-(4-fluorophenyl)-2-oxoethyl]pyrrolidin-3-yl}-2,2,2-trifluoroacetamide

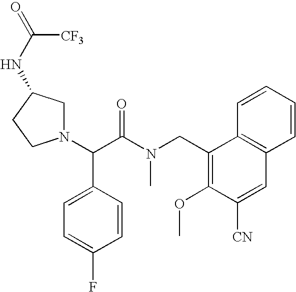

To the flask containing 2-[(3S)-3-aminopyrrolidin-1-yl]-N-[(3-cyano-2-methoxy-1-naphthyl)methyl]-2-(4-fluorophenyl)-N-methylacetamide (840 mg, 1.88 mmol) in DCM (12 mL) was added triethylamine (0.32 mL, 2.26 mmol) followed by trifluoroacetic anhydride (0.32 mL, 2.26 mmol) at 0° C. The mixture was stirred at room temperature over night. Solvent was evaporated. The reaction was neutralized with sat. aq. NaHCO₃ and extracted with EtOAc. The organic phase was dried over MgSO₄, filtered and concentrated. Chromatography of the residue on SiO₂ (0-1% MeOH:DCM) afforded the title compound (683 mg, 73%). MS m/z 543.3 (M+H)⁺. ¹H NMR (300.1 MHz, CDCl₃) δ 8.28-8.14 (m, 2H), 7.88-7.84 (m, 1H), 7.64-7.54 (m, 2H), 7.36-7.29 (m, 2H), 6.99 (t, 2H), 5.50-5.34 (m, 1H), 5.04-4.96 (m, 1H), 4.66-4.40 (m), 4.03 (d, 3H), 3.10-2.69 (m), 2.57 (d, 3H), 2.34-1.82 (m). The citrate salt was formed by the addition of citric acid (9 mg, 1.0 equivalent) to a methanolic solution of the title compound (26 mg). Concentration under reduced pressure afforded the desired salt form of the product as a white foam.

### Example 14

### N-{(3S)-1-[2-[[(3-cyano-2-methoxy-1-naphthyl)methyl](methyl)amino]l -(4-fluorophenyl)-2-oxoethyl]pyrrolidin-3-yl}-2,2,2-trifluoro-N-methylacetamide

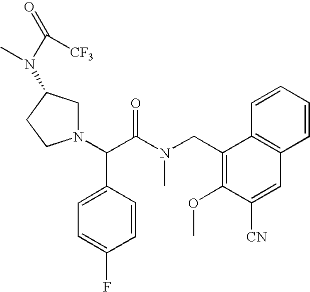

To the flask containing N-{(3S)-1-[2-[[(3-cyano-2-methoxy-1-naphthyl)methyl](methyl)amino]-1-(4-fluorophenyl)-2-oxoethyl]pyrrolidin-3-yl}-2,2,2-trifluoroacetamide (637 mg, 1.18 mmol) in DMF (12 mL) was added NaH (84 mg, 2.12 mmol, 60%) at 0° C. The mixture was stirred at room temperature for 15 min and cooled to 0° C. Iodomethane (0.095 mL, 1.53 mmol) was added. After stirring at room temperature over night, the reaction mixture was neutralized with sat. aq. NaHCO₃ and extracted with EtOAc. The organic phase was dried over MgSO₄, filtered and concentrated. Chromatography of the residue on SiO₂ (0-1% MeOH:DCM) afforded the title compound (438 mg, 67%). MS m/z 557.3 (M+H)⁺. ¹H NMR(300.1 MHz, CDCl₃) δ 8.19 (s, 1H), 8.11-7.96 (m, 1H), 7.83-7.81 (m, 1H), 7.54-7.40 (m, 4H), 6.98 (t, 2H), 5.26-5.06 (m), 4.60 (m), 4.21-4.09 (m), 4.01 (s, 3H), 3.30 (d, 1H), 3.15 (s), 3.04 (s, 1H), 2.75-1.88 (m).

### Example 15

### 2-{(3S)-3-[acetyl(methyl)amino]pyrrolidin-1-yl}-N-[(3-cyano-2-methoxy-1-naphthyl)methyl]-2-(4-fluorophenyl)-N-methylacetamide

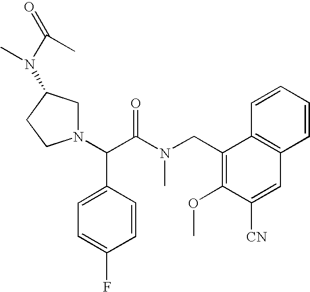

To the flask containing N-[(3-cyano-2-methoxy-1-naphthyl)methyl]-2-(4-fluorophenyl)-N-methyl-2-[(3S)-3-(methylamino)pyrrolidin-1-yl]acetamide (87 mg, 0.189 mmol) in DCM (5 mL) was added triethylamine (0.066 mL, 0.473 mmol) followed by acetyl chloride (0.016 mL, 0.227 mmol) at 0° C. The mixture was stirred at room temperature over night. The reaction was neutralized with sat. aq. NaHCO₃ and extracted with DCM. The organic phase was dried over MgSO₄, filtered and concentrated. Chromatography of the residue on SiO₂ (0-2% MeOH:DCM) afforded the title compound (73 mg, 77%). MS m/z 503.3 (M+H)⁺.¹1H NMR (300.1 MHz, CDCl₃) δ 8.18-7.93 (m, 2H), 7.82 (m, 1H), 7.53-7.39 (m, 4H), 6.97 (t, 2H), 5.25-5.04 (m), 4.48-4.10 (m), 4.01 (s, 3H), 3.21-2.87 (m), 2.74-2.36 (m), 2.23-2.08 (m), 1.87-1.70 (m). The citrate salt was formed by the addition of citric acid (28 mg, 1.0 equivalent) to a methanolic solution of the title compound (73 mg). Concentration under reduced pressure afforded the desired salt form of the product as a white foam.

The requisite N-[(3-cyano-2-methoxy-1-naphthyl)methyl]-2-(4-fluorophenyl)-N-methyl-2-[(3S)-3-(methylamino)pyrrolidin-1-yl]acetamide was synthesized using the following method.

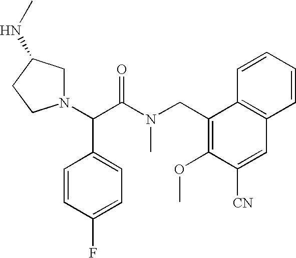

To the flask containing N-{(3S)-1-[2-[[(3-cyano-2-methoxy-1-naphthyl)methyl](methyl)amino]-1-(4-fluorophenyl)-2-oxoethyl]pyrrolidin-3-yl}-2,2,2-trifluoro-N-methylacetamide (407 mg, 0.732 mmol) in MeOH (20 mL) and water (1.2 mL) was added K₂CO₃ (525 mg, 3.806 mmol). The mixture was stirred at room temperature over night and solvent was evaporated. The reaction mixture was then partitioned in water/DCM and extracted with DCM. The organic phase was dried over MgSO₄, filtered and concentrated. Chromatography of the residue on SiO₂ (0-8% MeOH:DCM) afforded the title compound (263 mg, 78%). MS m/z 461.3 (M+H)⁺. ¹H NMR (300.1 MHz, CDCl₃) δ 8.18 (s, 1H), 8.09 (d, 1H), 7.83-7.80 (m, 1H), 7.56-7.48 (m, 2H), 7.43-7.38 (q, 2H), 6.95 (t, 2H), 5.23-5.08 (m, 2H), 4.20 (d, 2H), 4.00 (s, 3H), 3.25-3.22 (m), 2.98-2.93 (m), 2.76-2.05 (m), 1.65-1.57 (m)

### Example 16

### 2-[3-(acetylamino)azetidin-1-yl]-N-[(3-cyano-2-methoxy-1-naphthyl)methyl]-2-(4-fluorophenyl)-N-methylacetamide

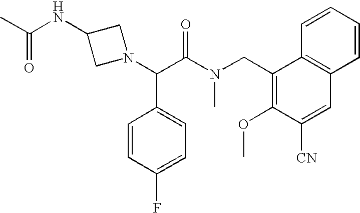

To the flask containing 2-(3-aminoazetidin-1-yl)-N-[(3-cyano-2-methoxy-1-naphthyl)methyl]-2-(4-fluorophenyl)-N-methylacetamide (114 mg, 0.264 mmol) in pyridine (1 mL) was added triethylamine (0.092 mL, 0.660 mmol) followed by acetic anhydride (0.05 mL, 0.528 mmol) at 0° C. The mixture was stirred at room temperature over night and CHCl₃ was added. After washed with sat. aq. NaHCO₃ and sat. aq. NaCl, the organic phase was dried over MgSO₄, filtered and concentrated. Chromatography of the residue on SiO₂ (0-2% MeOH:DCM) afforded the title compound (65 mg, 52%). MS m/z 475.2 (M+H)⁺. ¹H NMR (300.1 MHz, CDCl₃) δ 8.18 (s, 1H), 8.12 (d, 1H), 7.82 (d, 1H), 7.62-7.51 (m, 2H), 7.32 (q, 2H), 6.95 (t, 2H), 6.21 (m, 1H), 5.15 (d, 2H), 4.51 (m, 1H), 4.22 (s, 1H), 4.01 (s, 3H), 3.59 (t, 1H), 3.47 (t, 1H), 3.31 (m, 1H), 3.06 (m, 1H), 2.59 (s, 3H), 1.97 (s, 3H). The citrate salt was formed by the addition of citric acid (26 mg, 1.0 equivalent) to a methanolic solution of the title compound (65 mg). Concentration under reduced pressure afforded the desired salt form of the product as a white foam.

### Example 17

### methyl {[2-[[(3-cyano-2-methoxy-1-naphthyl)methyl](methyl)amino]1-(4-fluorophenyl)-2-oxoethyl]amino}acetate

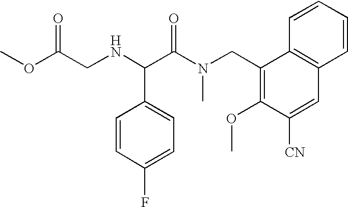

To the mixture of 2-amino-N-[(3-cyano-2-methoxy-1-naphthyl)methyl]-2-(4-fluorophenyl)-N-methylacetamide (334 mg, 0.885 mmol) in THF (3 mL) was added methyl bromoacetate (0.077 mL, 0.843 mmol) followed by triethylamine (0.124 mL. 0.885 mmol). The mixture was microwaved a 120° C. for 10 min. The mixture was filtered through a cotton plug to remove the salt and washed with EtOAc. Chromatography of the residue on SiO₂ (0-60% EtOAc:hexane) afforded the title compound (230 mg, 58%). MS m/z 450.2 (M+H)⁺. ¹H NMR (300.1 MHz, CDCl₃) δ 8.18 (s, 1H), 8.11 (d, 1H), 7.83 (d, 1H), 7.62-7.50 (m, 2H), 7.29 (q, 2H), 6.94 (t, 2H), 5.27-5.12 (q, 2H), 4.62 (s, 1H), 4.02 (s, 3H), 3.69 (s, 3H), 3.45-3.31 (q, 2H), 2.60 (s, 3H).

### Example 18

### N-[(3-cyano-2-methoxy-1-naphthyl)methyl]-2-(4-fluorophenyl)-2-[(2-hydroxyethyl)amino]N-methylacetamide

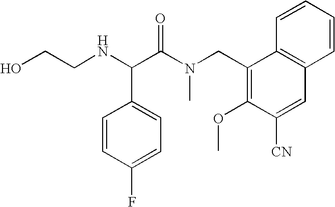

To the flask containing methyl {[2-[[(3-cyano-2-methoxy-1-naphthyl)methyl](methyl)amino]-1-(4-fluorophenyl)-2-oxoethyl]amino} acetate (133 mg, 0.296 mmol) in MeOH (6 mL) was added NaBH₄ (111 mg, 2.96 mmol) at room temperature. The mixture was stirred at room temperature for 3 h and water was added. The mixture was extracted with EtOAc and the organic phase was dried over MgSO₄, filtered and concentrated. Chromatography of the residue on SiO₂ (0-3% MeOH:DCM) afforded the title compound (55 mg, 44%). MS m/z 422.2 (M+H)⁺. ¹H NMR (300.1 MHz, CDCl₃) δ 8.20 (s, 1H), 7.84 (d, 1H), 7.62-7.51 (m, 2H), 7.31-7.26 (m, 2H), 6.96 (t, 2H), 5.27-5.14 (q, 2H), 4.47 (s, 1H), 4.03 (s, 3H), 3.61 (t, 2H), 2.85-2.78 (m, 1H), 2.70-2.66 (m, 1H), 2.63 (s, 3H). The citrate salt was formed by the addition of citric acid (25 mg, 1.0 equivalent) to a methanolic solution of the title compound (55 mg). Concentration under reduced pressure afforded the desired salt form of the product as a white foam.

### Example 19

### methyl[[2-[[(3-cyano-2-methoxy-1-naphthyl)methyl](methyl amino]1-(4-fluorophenyl-2-oxoethyl](methyl)amino]acetate

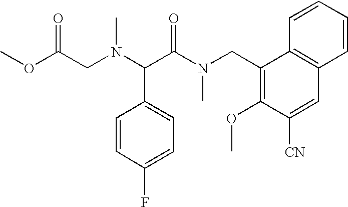

To the mixture of N-[(3-cyano-2-methoxy-1-naphthyl)methyl]-2-(4-fluorophenyl)-N-methyl-2-(methylamino)acetamide (80 mg, 0.204 mmol) in THF (2 mL) was added methyl bromoacetate (0.018 mL, 0.195 mmol) followed by triethylamine (0.028 mL. 0.204 mmol). The mixture was microwaved a 120° C. for 1 min. The mixture was filtered through a cotton plug to remove the salt and washed with EtOAc. Chromatography of the residue on SiO₂ (0-40% EtOAc:hexane) afforded the title compound (42 mg, 44%). MS m/z 464.2 (M+H)⁺. ¹H NMR (300.1 MHz, CDCl₃) δ 8.18 (s, 1H), 8.13-8.10 (m, 1H), 7.82 (dd, 1H), 7.60-7.40 (m, 4H), 6.97 (t, 2H), 5.32-4.98 (m, 3H), 4.02 (s, 3H), 3.68-3.64 (d, 1H), 3.58 (s, 3H), 3.38-3.32 (d, 1H), 2.69 (s, 3H), 2.49 (s, 3H). The citrate salt was formed by the addition of citric acid (17 mg, 1.0 equivalent) to a methanolic solution of the title compound (42 mg). Concentration under reduced pressure afforded the desired salt form of the product as a white foam.

### Example 20

### [[2-[[(3-cyano-2-methoxy-1-naphthyl)methyl](methyl)amino]-1-(4-fluorophenyl)-2-oxoethyl](methyl)amino]acetic acid

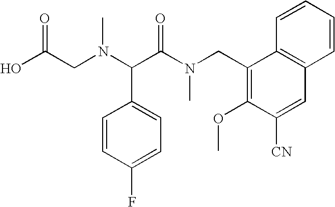

To the mixture of methyl [[2-[[(3-cyano-2-methoxy-1-naphthyl)methyl](methyl)amino]-1-(4-fluorophenyl)-2-oxoethyl](methyl)amino]acetate (38 mg, 0.058 mmol) in THF (3 mL) and water (1.5 mL) was added 1N NaOH (0.145 mL, 0.145 mmol). The mixture was stirred at room temperature for 4 h and adjusted to acidic by adding TFA. HPLC (Gilson) separation of the residue afforded the title compound as TFA salt (35 mg, 100%). MS m/z 450.2 (M+H)⁺. ¹H NMR (300.1 MHz, DMSO-d₆) δ 8.64 (t, 1H), 8.04 (d, 1H), 7.93 (d, 1H), 7.71 (t, 2H), 7.64-7.56 (m, 2H), 7.25 (t, 2H), 5.63 (s), 5.29-4.90 (dd, 2H), 3.97 (s, 3H), 2.69 (s, 2H), 2.55-2.50 (m).

### Example 21

### N-(3-Cyano-2-methoxy-naphthalen-1-ylmethyl)-2-(4-fluoro-phenyl)-N-methyl-2-((S)-3-morpholin-4-yl-pyrrolidin-1-yl)-acetamide

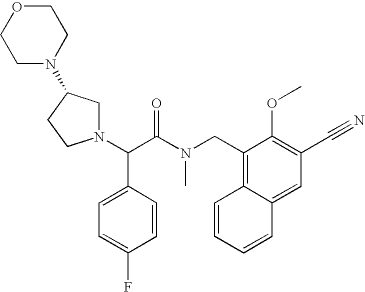

To a stirring slurry of 2-((S)-3-amino-pyrrolidin-1-yl)-N-(3-cyano-2-methoxy-naphthalen-1-ylmethyl)-2-(4-fluoro-phenyl)-N-methyl-acetamide (50 mg, 0.11 mmol) and potassium carbonate (46 mg, 0.33 mmol) in ACN (2.2 mL) was added diethylene glycol di(p-toluenesulfonate) (50 mg, 0.12 mmol). The mixture was subject to microwave radiation at 120 ° C. for 105 min before stirring at ambient temperature for 14 h. The resultant slurry was partitioned with water and extracted with DCM (3×15 mL), the organics being concentrated in vacuo to a residue which was then subject to flash chromatography (SiO₂; 4 g; gradient elution: 1% MeOH:DCM for 1 min, then 1-5% MeOH:DCM over 8 min at 20 mL/min) to provide the title compound as a colorless film (41 mg, 72%). MS m/z 517.30 (M+H)⁺. ¹H NMR (300 MHz, CDCl₃) δ 8.18 (s, 1H), 8.09 (m, 1H), 7.81 (m, 1H), 7.51 (m, 2H), 7.39-7.44 (m, 2H), 6.96 (t, J=8.6 Hz, 2H), 5.15 (app dd, J=21.4, 14.3 Hz, 2H), 4.19 (d, J=3.8 Hz, 1H), 4.01 (s, 3H), 3.68 (app t, J=4.2 Hz, 4H), 2.85-3.08 (m, 2H), 2.73 (app d, J=10.4 Hz, 3H), 2.60 (dd, J=13.9, 8.4 Hz, 1H), 2.44-2.48 (m, 3H), 2.25-2.36 (m, 3H), 1.95-2.09 (m, 2H), 1.71 (m, 1H). The citrate salt was generated by the addition of citric acid (15.2 mg, 1.0 equivalents) to a methanolic solution of the title compound (41 mg). Concentration under reduced pressure afforded the desired salt form of the product as a white foam. MS m/z 517.30 (M+H)⁺.

### Example 22

### N-(3-Cyano-2-methoxy-naphthalen-1-ylmethyl)-2-((R)-3-imethylamino-pyrrolidin-1-yl)-2-(4-fluoro-phenol)-N-methyl-acetamide

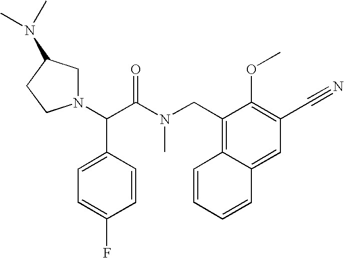 A solution of 2-((R)-3-amino-pyrrolidin-1-yl)-N-(3-cyano-2-methoxy-naphthalen 1-ylmethyl)-2-(4-fluoro-phenyl)-N-methyl-acetamide (65 mg, 0.15 mmol) and formaldehyde (37% aq., 61 μL, 0.73 mmol) in formic acid (0.5 mL) was heated via microwave radiation to 100° C. for 6 min. Upon cooling, the reaction was dilute with DCM, neutralized with dilute aqueous potassium carbonate then extracted with DCM (3×15 mL). The combined organics were dried over Na₂SO₄, filtered and concentrated under reduced pressure wherein the resultant residue was subject to flash chromatography (SiO₂; 4 g; gradient elution: 1-5% 2N NH₃-MeOH:DCM over 9 min at 20 mL/min) to afford the N-(3-cyano-2-methoxy-naphthalen-1-ylmethyl)-2-((R)-3-imethylamino-pyrrolidin-1-yl)-2-(4-fluoro-phenyl)-N-methyl-acetamide as a colorless film (57 mg, 83%). MS m/z 475.25 (M+H)⁺. ¹H NMR (300 MHz, CDCl₃) δ 8.17 (s, 1H), 8.09 (app t, J=3.9 Hz, 1H), 7.82 (m, 1H), 7.52 (m, 2H), 7.43 (dd, J=6.9, 5.6 Hz, 2H), 6.96 (app td, J=8.6, 2.0 Hz, 2H), 5.09-5.22 (m, 2H), 4.18 (s, 1H), 4.00 (s, 3H), 2.97-3.15 (m, 1H), 2.75-2.88 (m, 1H), 2.74 (app d, J=8.2 Hz, 3H), 2.20-2.62 (m, 2H), 2.16 (app d, J=5.0 Hz, 6H), 1.92-2.09 (m, 2H), 1.69 (ddd, J=27.1, 12.9, 5.8 Hz, 1H). The citrate salt was formed by the addition of citric acid (23.1 mg, 1.0 equivalents) to a methanolic solution of the title compound (57 mg). Concentration under reduced pressure afforded the desired salt form of-the product as a white film. MS m/z 475.25 (M+H)⁺.

### Example 23

### 2-(R)-[1,3′]Bipyrrolidini-1′-yl-N-(3-cyano-2-methoxy-naphthalen-1-ylmethyl)-2-(4-fluoro-phenyl)-N-methyl-acetamide

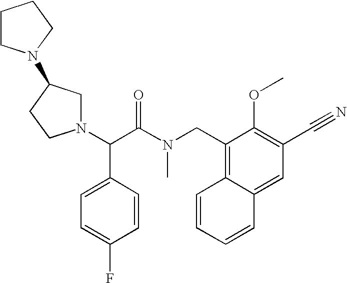

To a stirring slurry of 2-((R)-3-amino-pyrrolidin-1-yl)-N-(3-cyano-2-methoxy-naphthalen-1-ylmethyl)-2-(4-fluoro-phenyl)-N-methyl-acetamide (77 mg, 0.17 mmol) and potassium carbonate (74 mg, 0.54 mmol) in ACN (4 mL) was added 1,4-dibromobutane (22 μL, 0.18 mmol). The mixture was subject to microwave radiation at 150° C. for 120 min. The mixture was dilute with water, extracted with DCM (3×15 mL), dried over Na₂SO₄, filtered, and concentrated under reduced pressure to a red-orange gum. The resultant was subject to flash chromatography (SiO₂; 4 g; gradient elution: 1-5% 2N NH3-MeOH:DCM over 12 min at 20 mL/min) giving rise to the title compound as a colorless film (56 mg, 66%). MS m/z 501.26 (M+H)⁺. ¹H NMR (300 MHz, CDCl₃) δ 8.17 (s, 1H), 8.9 (m, 1H), 7.81 (m, 1H), 7.48-7.52 (m, 2H), 7.40-7.44 (m, 2H), 6.95 (td, J=8.6, 2.0 Hz, 2H), 5.15 (m, 2H), 4.18 (s, 1H), 4.00 (s, 3H), 3.04-3.24 (m, 1H), 2.76-2.95 (m, 1H), 2.73 (app d, J=8.2 Hz, 3H), 2.60 (td, J=8.4, 4.4 Hz, 1H), 2.38-2.49 (m, 4H), 2.23 (td, J=7.9, 3.2 Hz, 1H), 1.95-2.17 (m, 2H), 1.74-1.83 (m, 5H). The citrate salt was produced by the addition of citric acid (21.4 mg, 1.0 equivalents) to a methanolic solution of the title compound (56 mg). Concentration under reduced pressure afforded the desired salt form of the product as a white foam. MS m/z 501.26 (M+H)⁺.

### Example 24

### N-(3-Cyano-2-methoxy-naphthalen-1-ylmethyl)-2-(4-fluoro-phenyl)-2-{(R)-3-[(2-methoxy-acetyl)-methyl-amino]pyrrolidin-1-yl}-N-methyl-acetamide

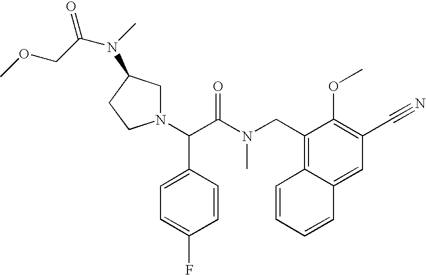

{(R)-1-[[(3-Cyano-2-methoxy-naphthalen-1-ylmethyl)-methyl-carbamoyl]-(4-fluoro-phenyl)-methyl]-pyrrolidin-3-yl}-methyl-carbamic acid tert-butyl ester (58 mg, 0.10 mmol) was deprotected via stirring in a TFA:DCM solution (ca. 1:1, 3 mL) at ambient temperature for 30 minutes before being concentrated by rotary evaporation. The residue was dissolved with DCM, washed with saturated aqueous potassium carbonate then added directly to a previously prepared (10 min prior to addition) stirring mixture of methoxyacetic acid (12 μL, 0.15 mmol) and polystyrene-supported carbodiimide (167 mg, 0.21 mmol) in DCM (3 mL). The mixture was left to stir overnight at ambient temperature. The reaction mixture was filtered through a 0.71 μm GMF filter, washing with DCM (3×3 mL, w/5 minute agitation per wash) then concentrated in vacuo. The resultant residue was subject to flash chromatography (SiO₂; 4 g; gradient elution: 0.5-5% MeOH:DCM over 10 min at 20 mL/min) to afford the title compound as a clear film (50 mg, 90%). MS m/z 533.3 (M+H)⁺. ¹H NMR (300 MHz, CDCl₃) δ 8.19 (s, 1H), 7.93-8.06 (m, 1H), 7.83 (m, 1H), 7.49-7.54 (m, 1H), 7.40-7.45 (m, 2H), 6.95-7.01 (m, 2H), 5.04-5.26 (m, 3H), 4.10 (s, 1H), 4.01 (s, 3H), 3.38-3.46 (m, 4H), 2.97 (s, 3H), 2.60-2.74 (m, 5H), 2.40-2.55 (m, 2H), 2.18 (m, 2H), 1.66-1.88 (m, 1H). The citrate salt was formed by the addition of citric acid (17.9 mg, 1.0 equivalents) to a methanolic solution of the title compound (50 mg). Concentration under reduced pressure afforded the desired salt form of the product as a white foam. MS m/z 533.3 (M+H)⁺.

The requisite {(R)-1-[[(3-Cyano-2-methoxy-naphthalen-1-ylmethyl)-methyl-carbamoyl]-(4-fluoro-phenyl)-methyl]-pyrrolidin-3-yl}-methyl-carbamic acid tert-butyl ester was prepared as follows.

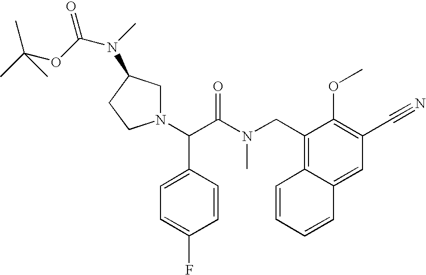

To anhydrous DMF (1 mL) under an argon gas atmosphere at 0° C. was added sodium hydride (27 mg, 1.13 mmol). After stirring for ca. 10 min a DMF (2 mL) solution of {(R)-1-[[(3-Cyano-2-methoxy-naphthalen-1-ylmethyl)-methyl-carbamoyl]-(4-fluoro-phenyl)-methyl]-pyrrolidin-3-yl}-carbamic acid tert-butyl ester (475 mg, 0.87 mmol) was added and the resultant orange-tan solution allowed to warm to 10° C. over 1 h. The solution was again cooled to 0° C. wherein methyl iodide (59 μL, 0.95 mmol) was added in one portion and the reaction allowed to warm to ambient temperature. After ca. 17 h the reaction was quenched with saturated aqueous ammonium chloride and extracted with DCM (3×40 mL), dried over Na₂SO₄, filtered, and concentrated in vacuo. The residue was subject to flash chromatography (SiO₂; 12 g; gradient elution: 30-85% EtOAc:Hexane; over 16 min at 40 mL/min) to afford the {(R)-1-[[(3-Cyano-2-methoxy-naphthalen-1-ylmethyl)-methyl-carbamoyl]-(4-fluoro-phenyl)-methyl]-pyrrolidin-3-yl}-methyl-carbamic acid tert-butyl ester as a clear film (116 mg, 24%). MS m/z 561.30 (M+H)⁺.¹H NMR (300 MHz, CDCl₃) δ 8.19 (s, 1H), 8.05 (m, 1H), 7.83 (t, J=3.8 Hz, 1H), 7.51 (app dd, J=9.3, 4.2 Hz, 2H), 7.43 (app dd, J=8.3, 5.5 Hz, 2H), 6.97 (t, J=8.3 Hz, 2H), 5.12 (m, 2H), 4.77 (s, 1H), 4.13 (m, 1H), 4.01 (s, 3H), 2.84 (app d, J=20.7 Hz, 3H), 2.74 (d, J=8.1 Hz, 3H), 2.63-2.46 (m, 3H), 2.22-2.05 (m, 2H), 1.83-1.70 (m, 1H), 1.45 (s, 9H).

### Example 25

### N-[(3-Cyano-2-methoxy-1-naphthyl)methyl]-N-methyl-2-phenyl-2-pyrrolidin-1-ylacetamide

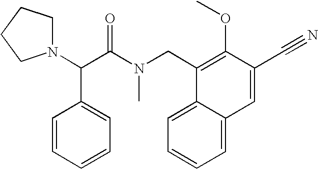

The title compound was synthesized using the similar procedure described for Example 3. MS m/z 414.1 (M+H)⁺. ¹H NMR (300 MHz, CDCl₃) δ 8.16 (s, 1H), 8.14 (d, J=11.3 Hz, 1H), 7.80 (dd, J=7.3, 1.7 Hz, 1H), 7.53 (1, 2H), 7.46 (d, J=6.0 Hz, 2H), 7.30-7.21 (m, 3H), 5.24 (d, J=14.0 Hz, 1H), 5.08 (d, J=14.0 Hz, 1H), 4.14 (s, 1H), 3.99 (s, 3H), 2.72 (s, 3H), 2.68-2.62 (m, 2H), 2.42-2.36 (m, 2H), 1.84-1.74 (m, 4H).

### Example 26

### Separation of enantiomers of N-[(3-cyano-2-methoxy-1-naphthyl)methyl]-N-methyl-2-phenyl-2-pyrrolidin-1-ylacetamide

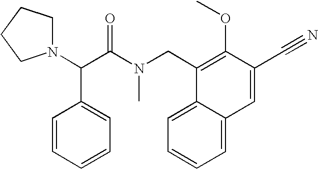

Individual enantiomers of N-(3-cyano-2-methoxy-naphthalen-1-ylmethyl)-N-methyl-2-phenyl-2-pyrrolidin-1-yl-acetamide were obtained by supercritical fluid chromatography (SFC-Berger Instruments) employing a ChiralPak AD-H column (21 mm×250 mm, 5 μm), 15% methanol with 0.5% dimethylethylamine additive:CO₂ isocratic at 50 mL/min at 35° C. Enantiomeric excess (ee) of each isomer was determined via SFC employing a ChiralPak AD-H column (4.6 mm×150 mm, 5 μm), 15% methanol with 0.5% dimethylethylamine additive:CO₂ isocratic at 2.2 mL/min at 35° C. over 7 min. Isomer 1: T_{R}=5.91 min; >99% ee. Isomer 2: T_{R}=6.64 min; 99% ee.

### Example 27

### 2-[(3S)-3-aminopyrrolidin-1-yl]-N-[(3-cyano-2-methoxy-1-naphthyl)methyl]-2-(4-fluorophenyl)-N-methylacetamide

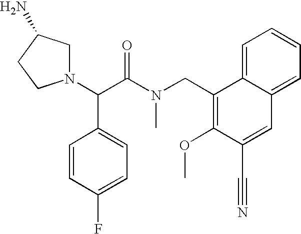

2-[(3S)-3-Aminopyrrolidin-1-yl]-N-[(3-cyano-2-methoxy-1-naphthyl)methyl]-2-(4-fluorophenyl)-N-methylacetamide was prepared in a manner analogous to that described in Example 2 using tert-butyl (3S)-pyrrolidin-3-ylcarbamate in place of tert-butyl piperazine-1-carboxylate.

### Example 28

### Separation of the diastereomers of 2-[(3S)-3-aminopyrrolidin-1-yl]-N-[(3-cyano-2-methoxy-1-naphthyl)methyl]-2-(4-fluorophenyl)-N-methylacetamide

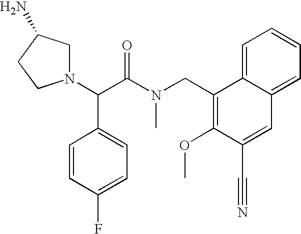

The diastereomeric mixture of 2-[(3S)-3-aminopyrrolidin-1-yl]-N-[(3-cyano-2-methoxy-1-naphthyl)methyl]-2-(4-fluorophenyl)-N-methylacetamide was separated using preparative supercritical fluid chromatography on a Berger Cyano column (20×150 mm, 6 μm) with an eluent consisting of 9% methanol containing 0.5% dimethylethylamine and carbon dioxide at a flow rate of 50 mL/min with detection at 280 nm. Diastereomeric purity was assessed by analysis with supercritical fluid chromatography on a Berger Cyano column (4.6×150 mm, 6 μm) with an eluent consisting of 9% methanol containing 0.5% dimethylethylamine and carbon dioxide at a flow rate of 3.1 mL/min with detection at 280 nm. Diastereomer Isomer 1: T_{R}=4.97 min; 98% de. MS m/z 447.2 (M+H). ¹H NMR (300.132 MHz, DMSO) δ 8.59 (s, 1H), 8.03-8.00 (m, 1H), 7.94-7.91 (m, 1H), 7.59 -7.56 (m, 2H), 7.48 (dd, J=8.2, 5.8 Hz, 2H), 7.11 (t, J=8.7 Hz, 2H), 5.02 (dd, J=36.9, 14.2 Hz, 2H), 4.44 (s, 1M), 3.94 (s, 3H), 2.69 (s, 3H), 2.64-2.55 (m, 4H), 2.17 (s, 1H), 2.01-1.91 (m, 1H), 1.39-1.30 (m, 1H). The citrate salt was formed by the addition of citric acid (1.0 equivalents) to a methanolic solution of the title compound. Concentration under reduced pressure afforded the desired salt form of the product as an off-white solid. Diastereomer Isomer 2: T_{R}=6.01 min; 94% de. MS m/z 447.2 (M+H). 1H NMR (300.132 MHz, DMSO) δ 8.59 (s, 1H), 8.03-8.00 (m, 1H), 7.93-7.90 (m, 1H), 7.58-7.56 (m, 2H), 7.51-7.46 (m, 2H), 7.11 (t, J=8.8 Hz, 2H), 5.02 (dd, J=31.0, 14.2 Hz, 2H), 4.44 (s, 1H), 3.94 (s, 3H), 2.78-2.60 (m, 2H), 2.70 (s, 3H), 2.45-2.41 (m, 1H), 2.37-2.33 (m, 1H), 2.18 -2.10 (m, 1H), 2.00-1.91 (m, 1H), 1.42-1.34 (m, 1H). The citrate salt was formed by the addition of citric acid (1.0 equivalents) to a methanolic solution of the title compound. Concentration under reduced pressure afforded the desired salt form of the product as an off-white solid.

### Example 29

### 2-[(3R)-3-aminopyrrolidin-1-yl]-N-[(3-cyano-2-methoxy-1-naphthyl)methyl]-2-(4-fluorophenyl)-N-methylacetamide

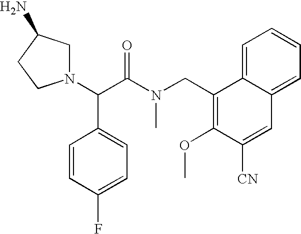

2-[(3R)-3-Aminopyrrolidin-1-yl]-N-[(3-cyano-2-methoxy-1-naphthyl)methyl]-2-(4-fluorophenyl)-N-methylacetamide was prepared in a manner analogous to that described in Example 2 using tert-butyl (3R)-pyrrolidin-3-ylcarbamate in place of tert-butyl piperazine-1-carboxylate.

### Example 30

### Separation of the diastereomers of 2-[(3R)-3-aminopyrrolidin-1-yl]-N-[(3-cyano-2-methoxy-1-naphthyl)methyl]-2-(4-fluorophenyl)-N-methylacetamide

A diastereomeric mixture of 2-[(3R)-3-aminopyrrolidin-1-yl]-N-[(3-cyano-2-methoxy-1-naphthyl)methyl]-2-(4-fluorophenyl)-N-methylacetamide was separated using preparative supercritical fluid chromatography and the diastereomeric purity was assessed as described in Example 28. Diastereomer Isomer 1: T_{R}=5.07 min; 98% de. MS m/z 447.1 (M+H). ¹H NMR (300.132 MHz, DMSO) δ 8.59 (s, 1H), 8.03-8.00 (m, 1H), 7.93-7.91 (m, 1H), 7.59-7.56 (m, 2H), 7.48 (t, J=6.9 Hz, 2H), 7.11 (t, J=8.7 Hz, 2H), 5.02 (dd, J=37.2, 14.2 Hz, 2H), 4.44 (s, 1H), 3.94 (s, 3H), 2.69 (s, 3H), 2.64-2.55 (m, 4H), 2.21-2.14 (m, 1H), 2.02-1.90 (m, 1H), 1.40-1.29 (m, 1H). The citrate salt was formed by the addition of citric acid (1.0 equivalents) to a methanolic solution of the title compound. Concentration under reduced pressure afforded the desired salt form of the product as an off-white solid. Diastereomer Isomer 2: T_{R}=6.17 min, 95% de. MS m/z 447.2 (M+H). ¹H NMR (300.132 MHz, DMSO) δ 8.59 (s, 1H), 8.03-8.00 (m, 1H), 7.91 (d, J=8.8 Hz, 1H), 7.58-7.56 (m, 2H), 7.51-7.46 (m, 2H), 7.11 (t, J-8.8 Hz, 2H), 5.02 (dd, J=31.3, 14.2 Hz, 2H), 4.44 (s, 1H), 2.78-2.60 (m, 6H), 2.70 (s, 3H), 2.36-2.34 (m, 1H), 2.17-2.10 (m, 1H), 1.96-1.89 (m, 1H), 1.41-1.33 (m, 1H). The citrate salt was formed by the addition of citric acid (1.0 equivalents) to a methanolic solution of the title compound. Concentration under reduced pressure afforded the desired salt form of the product as an off-white solid.

### Example 31

### N-{2-[3,5-bis(trifluoromethyl)phenyl]ethyl}-2-(4-fluorophenyl)-N-methyl-2-moripholin-4-ylacetamide

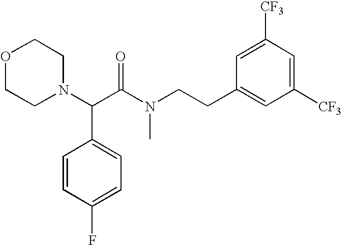

The title compound was synthesized using the similar procedure described for Example 3. MS m/z 493.2 (M+H). ¹H NMR (300.132 MHz, DMSO) δ 7.95 (s, 1H), 7.89 (s, 2H), 7.42-7.32 (m, 2H), 7.17-7.06 (m, 2H), 4.31-4.28 (m, 1H), 3.82-3.71 (m, 1H), 3.58-3.52 (m, 1H), 3.43 (s, 4H), 2.97 (t, J=6.8 Hz, 1H), 2.93 (s, 3H), 2.86 (s, 1H), 2.21-2.11 (m, 4H).

### Example 32

### Separation of the enantiomers of N-{2-[3,5-bis(trifluoromethyl)-phenyl]ethyl}-2-(4-fluorophenyl)-N-methyl-2-morpholin-4-ylacetamide

Preparative Chromatography on N-{2-[3,5-bis(trifluoromethyl)phenyl]ethyl}-2-(3-methoxyphenyl)-N-methyl-2-morpholin-4-ylacetamide was carried out on a Chiralpak AD column (20×250 mm, 10 μm) using a mobile phase of 2% isopropanol and hexane at a flow rate of 20 mL/min with detection at 210 nm. A stock solution of the racemate was prepared at 100 mg/5 mL in 0.2 mL DCM, 0.2 mL isopropanol and 4.6 mL of hexane. Sample injections were 2.5 mL of the stock solution (50 mg/injection). Chiral purity was assessed by analysis on a Chiralpak AD column (4.6×250 mm, 10 μm) using a mobile phase of 2% isopropanol and hexane at a flow rate of 1.0 mL/min with detection at 210 nm. Isomer 1: T_{R}=14.42 min; >99% ee. Isomer 2: T_{R}=18.82 min; >99% ee.

### Example 33

### N-{2-[3,5-bis(trifluoromethyl)phenyl]ethyl}-2-(3-methoxyphenyl)-N-methyl-2-morpholin-4-ylacetamide

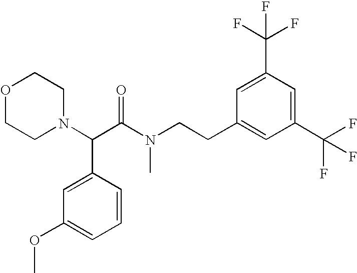

The title compound was prepared in a manner analogous to that described in Example 3. MS m/z 505.1 (M+H)⁺. ¹H NMR (300.132 MHz, DMSO) δ 7.94-7.89 (m, 3H), 7.28-7.17 (m, 1H), 6.95-6.83 (m, 3H), 4.24-4.21 (m, 1H), 3.71 (s, 3H), 3.43 (br s, 4H), 2.99-2.85 (m, 5H), 2.19-2.07 (m, 6H).

### Example 34

### Separation of the enantiomers of N-{2-[3,5-bis(trifluoromethyl)-phenyl]ethyl}-2-(3-methoxyphenyl)-N-methyl-2-morpholin-4-ylacetamide

Preparative Chromatography on N-{2-[3,5-bis(trifluoromethyl)phenyl]ethyl}-2-(3-methoxyphenyl)-N-methyl-2-morpholin-4-ylacetamide was carried out on a Chiralpak AD column (20×250 mm, 10 μm) using a mobile phase of 2% isopropanol and hexane at a flow rate of 20 mL/min with detection at 210 nm. A stock solution of the racemate was prepared at 100 mg/5 mL in 0.2 mL DCM, 0.2 mL isopropanol and 4.6 mL of hexane. Sample injections were 2.5 mL of the stock solution (50 mg/injection). Chiral purity was assessed by analysis on a Chiralpak AD column (4.6×250 mm, 10 μm) using a mobile phase of 2% isopropanol and hexane at a flow rate of 1.0 mL/min with detection at 210 nm. Isomer 1: T_{R}=16.44 min; >99% ee. Isomer 2: T_{R}=18.92 min; >99% ee.

### Example 35

### N-[(3-cyano-2-methoxy-1-naphthyl)methyl]-2-(4-fluorophenyl)-N-methyl-2-morpholin-4-ylacetamide

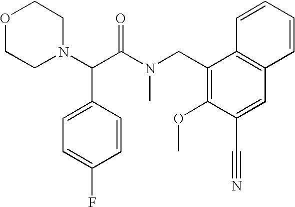

The title compound was prepared in a manner analogous to that described in Example 3. MS m/z 448.1 (M+H)⁺. ¹H NMR (300.132 MHz, DMSO) δ 8.59 (s, 1H), 8.03-8.00 (m, 1H), 7.92-7.89 (m, 1H), 7.60-7.55 (m, 2H), 7.50-7.45 (m, 2H), 7.13 (t, J=8.8 Hz, 2H), 5.03 (s, 2H), 4.50 (s, 1H), 3.95 (s, 3H), 3.53 (br s, 4H), 2.70 (s, 3H), 2.43-2.41 (m, 4H).

### Example 36

### Separation of the enantiomers of N-[(3-cyano-2-methoxy-1-naphthyl)methyl]-2-(4-fluorophenyl)-N-methyl-2-morpholin-4-ylacetamide

Preparative Chromatography on N-[(3-cyano-2-methoxy-1-naphthyl)methyl]-2-(4-fluorophenyl)-N-methyl-2-morpholin-4-ylacetamide was carried out on a Chiralpak AD column (20×250 mm, 10 μm) using a mobile phase of 10% isopropanol and hexane at a flow rate of 20 mL/min with detection at 210 nm. A stock solution of the racemate was prepared at 200 mg/10 mL in 0.4 mL DCM, 1.0 mL isopropanol and 8.6 mL of hexane. Sample injections were 2.5 mL of the stock solution (50 mg/injection). Chiral purity was assessed by analysis on a Chiralpak AD column (4.6×250 mm, 10 μm) using a mobile phase of 10% isopropanol and hexane at a flow rate of 1.0 mL/min with detection at 210 nm. Isomer 1: T_{R}=9.56 min; >99% ee. Isomer 2: T_{R}=11.71 min; >99% ee.

### Example 37

### N-[(3-cyano-2-methoxy-1-naphthyl)methyl]-2-(dimethylamino)-2-(4-fluorophenyl)-N-methylacetamide

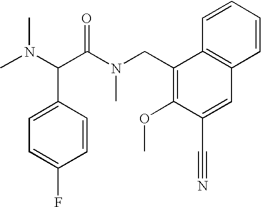

The TFA salt of (dimethylamino)(4-fluorophenyl)acetic acid (273 mg, 0.877 mmol), 3-methoxy-4-[(methylamino)methyl]-2-naphthonitrile (198 mg, 0.877 mmol), HOBT (130 mg, 0.964 mmol), 1-(3-dimethylaminopropyl)-3-ethylcarbodiimide hydrochloride (202 mg, 1.05 mmol) and diisopropylethylamide (0.61 mL, 3.50 mmol) were reacted together in DCM (20 mL) overnight. The reaction mixture was partitioned between water (20 mL) and DCM (20 mL). The organic layer was washed with sat. aq. NaHCO₃, dried over Na₂SO₄, filtered through a pad of diatomaceous earth and the volatiles were removed under reduced pressure. Chromatography of the residue on SiO₂ (12 g; 0-5% MeOH:DCM) afforded the title compound as a white solid (230 mg, 64%). MS m/z 406.4 (M+H)⁺. ¹H NMR (300.132 MHz, DMSO) δ 8.60 (s, 1H), 8.03-7.94 (m, 2H), 7.59-7.56 (m, 2H), 7.48-7.44 (m, 2H), 7.13 (t, J=8.7 Hz, 2H), 5.04 (dd, J=41.1, 14.1 Hz, 2H), 4.42 (s, 1H), 3.95 (s, 3H), 2.69 (s, 3H), 2.18 (s, 6H).

### Example 38

### Chiral separation of the enantiomers of N-[(3-cyano-2-methoxy-1-naphthyl)methyl]-2-(dimethylamino)-2-(4-fluorophenyl)-N-methylacetamide

A racemic mixture of N-[(3-cyano-2-methoxy-1-naphthyl)methyl]-2-(dimethylamino) -2-(4-fluorophenyl)-N-methylacetamide was separated into its component enantiomers using preparative supercritical fluid chromatography on a Chiralpak AD-H column (20×250 mm, 5 μm) with an eluent consisting of 12% methanol containing 0.5% dimethylethylamine and carbon dioxide at a flow rate of 50 mL/min with detection at 280 nm. Chrial purity was assessed by analysis with supercritical fluid chromatography on a Chiralpak AD-H column (4.6×250 mm, 5 μm) with an eluent consisting of 12% methanol containing 0.5% dimethylethylamine and carbon dioxide at a flow rate of 2.2 mL/min. with detection at 280 nm. Isomer 1: T_{R}=4.86 min; >99% ee. Isomer 2: T_{R}=5.41 min; 94% ee.

The requisite (dimethylamino)(4-fluorophenyl)acetic acid was synthesized using the following method.

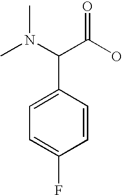

Dimethylamine (2.0 M, THF, 5.54 mL, 11.08 mmol), (4-fluorophenyl)boronic acid (1.55 g, 11.08 mmol) and glyoxylic acid monohydrate (1.02 g, 11.08 mmol) were reacted together in DCM (20 mL) at reflux overnight. The volatiles were removed under reduced pressure. The resulting residue was taken up in EtOAc (10 mL) and H₂O (10 mL). The layers were separated and the aqueous layer was concentrated under reduced pressure. Preparative Chromatography was carried out on a Dynamax C-18 column (21.4×250 mm, 8 μm, 60 Å) using a mobile phase of 2% ACN with 0.1% TFA:H₂O with 0.1% TFA at a flow rate of 20 mL/min with detection at 210 nm. A stock solution of the residue was prepared at 2.0 g/10 mL MeOH. Sample injections were 2 mL of the stock solution (400 mg/injection). Concentrated of the fractions under reduced pressure followed by chasing with ether afforded the desired TFA salt product as a white foam (2.3 g, 67%). MS m/z 198.1 (M+H)⁺.¹H NMR (300.132 MHz, DMSO) δ 7.54 (dd, J=8.7, 5.4 Hz, 2H), 7.35 (dd, J=8.8, 8.8 Hz, 2H), 5.05 (s, 1H), 2.67 (s, 6H).

### Example 39

### N-[(3-cyano-2-methoxy-1-naphthyl)methyl]-2-(4-fluorophenyl)-N-methyl-2-[(3S)-3-(methylamino)pyrrolidin-1-yl]acetamide (isomer 2)

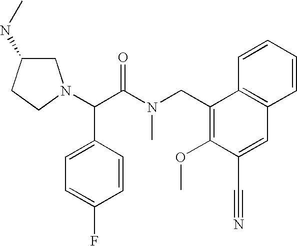

2-[(3S)-3-aminopyrrolidin-1-yl]-N-[(3-cyano-2-methoxy-1-naphthyl)methyl]-2-(4-fluorophenyl)-N-methylacetamide (65 mg, 0.146 mmol; Isomer 2 from Example 28) was dissolved in MeOH (5mL) and NaOMe (39 mg, 0.73 mmol) was added in one portion and paraformaldehyde (6.1 mg, 0.204 mmol) was added. The reaction mixture was stirred at 70° C. for 5 h under nitrogen. Next, the reaction was removed from the oil bath and NaBH₄ was added in one portion and reaction mixture heated at 70° C. overnight. The cooled reaction was neutralized with 1 N NaOH (2 mL) and extracted with DCM. . The organic phase was dried over Na₂SO₄, filtered through a pad of diatomaceous earth and the volatiles removed under reduce pressure. Chromatography of the residue on SiO₂ (0-5% MeOH:DCM) afforded the title compound (21 mg, 31%). Diastereomeric purity was assessed by analysis with supercritical fluid chromatography on a Chiralpak AD-H column (4.6×250 mm, 5 μm) with an eluent consisting of 17% methanol containing 0.5% dimethylethylamine and carbon dioxide at a flow rate of 2.2 mL/min with detection at 280 nm. T_{R}=5.33 min; 94% de. MS m/z 461.2 (M+H)⁺. ¹H NMR (300.132 MHz, DMSO) δ 8.59 (s, 1H), 8.03-8.00 (m, 1H), 7.93-7.90 (m, 1H), 7.58-7.55 (m, 2H), 7.51-7.46 (m, 2H), 7.11 (t, J=8.9 Hz, 2H), 5.02 (dd, J=36.7, 14.3 Hz, 2H), 4.43 (s, 1H), 3.94 (s, 3H), 3.10-3.05 (m, 1H), 2.84-2.78 (m, 1H), 2.71 (s, 3H), 2.52-2.50 (m, 1H) 2.45-2.31 (m, 2H), 2.18 (s, 3H), 1.93-1.81 (m, 1H), 1.50-1.36 (m, 1H).

### Example 40

### N-[(3-cyano-2-methoxy-1-naphthyl)methyl]-2-(4-fluorophenyl)-N-methyl-2-[(3S)-3-(methylamino)pyrrolidin-1-yl]acetamide (isomer 1)

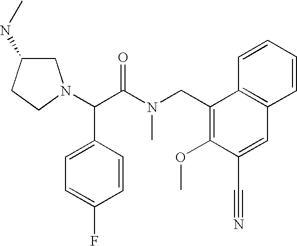

The title compound was prepared as describe for Example 39 using Isomer 1 from Example 28. T_{R}=3.62 min; 74% de. MS m/z 461.2 (M+H)⁺. ¹H NMR (300.132 MHz, DMSO) δ 8.59 (s, 1H), 8.02-8.00 (m, 1H), 7.94-7.91 (m, 1H), 7.58-7.56 (m, 2H), 7.51-7.46 (m, 2H), 7.11 (t, J=8.7 Hz, 2H), 5.02 (dd, J=35.0, 14.1 Hz, 2H), 4.43 (s, 1H), 3.94 (s, 3H), 3.08-3.02 (m, 1H), 2.70 (s, 3H), 2.62-2.55 (m, 2H), 2.45-2.42 (m, 1H), 2.33-2.28 (m, 1H), 2.20 (s, 3H), 1.95-1.91 (m, 1H), 1.44-1.42 (m, 1H).

### Example 41

### N-[(3-cyano-2-methoxy-1-naphthyl)methyl]-2-(4-fluorophenyl)-N-methyl-2-[(3R)-3-(methylamino)pyrrolidin-1-yl]acetamide (isomer 1)

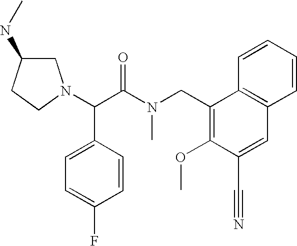

The title compound was prepared as describe for Example 39 using Isomer 1 from Example 30. T_{R}=4.61 min; 63% de. MS m/z 461.2 (M+H)⁺. ¹H NMR (300.132 MHz, DMSO) δ 8.59 (s, 1H), 8.03-8.00 (m, 1H), 7.94-7.91 (m, 1H), 7.58-7.56 (m, 2H), 7.51-7.46 (m, 2H), 7.11 (t, J=8.8 Hz, 2H), 5.02 (dd, J=35.2, 14.2 Hz, 2H), 4.43 (s, 1H), 3.94 (s, 3H), 3.07-3.02 (m, 1H), 2.70 (s, 3H), 2.61-2.56 (m, 2H), 2.44-2.42 (m, 1H), 2.32-2.27 (m, 1H), 2.19 (s, 3H), 1.95-1.89 (m, 1H), 1.43-1.41 (m, 1H).

### Example 42

### N-[(3-cyano-2-methoxy-1-naphthyl)methyl]-2-(4-fluorophenyl)-N-methyl-2-[(3R)-3-(methylamino)pyrrolidin-1-yl]acetamide (isomer 2)

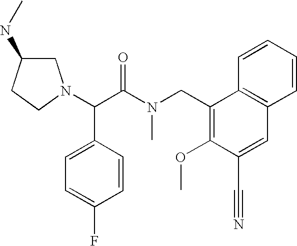

The title compound was prepared as describe for Example 39 using Isomer 2 from Example 30. T_{R}=4.43 min; 77% de. MS m/z 461.2 (M+H)⁺. ¹H NMR (300.132 MHz, DMSO) δ 8.59 (s, 1H), 8.03-8.00 (m, 1H), 7.93-7.90 (m, 1H), 7.58-7.55 (m, 2H), 7.51-7.46 (m, 2H), 7.11 (t, J=8.8 Hz, 2H), 5.02 (dd, J=36.7, 14.3 Hz, 2H), 4.43 (s, 1H), 3.94 (s, 3H), 3.10-3.06 (m, 1H), 2.84-2.78 (m, 1H), 2.71 (s, 3H), 2.57-2.53 (m, 2H), 2.38-2.35 (m, 1H), 2.18 (s, 3H), 1.91-1.84 (m, 1H), 1.45-1.44 (m, 1H).

### Example 43

### N-[(3-cyano-2-methoxy-1-naphthyl)methyl]-2-(4-fluorophenyl)-N-methyl-2-(4-methylpiperazin-1-yl)acetamide

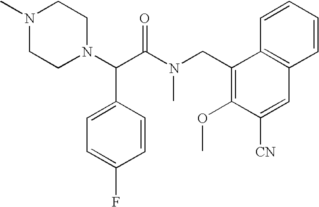

The title compound was prepared in a manner analogous to that described in Example 1 using chiral N-[(3-cyano-2-methoxy-1-naphthyl)methyl]-2-(4-fluorophenyl)-N-methyl-2-piperazin-1-ylacetamide. MS m/z 461.1 (M+H)⁺. ¹H NMR (300.132 MHz, DMSO) δ 8.60 (s, 1H), 8.01 (d, J=7.7 Hz, 1H), 7.89 (d, J=8.2 Hz, 1H), 7.60-7.53 (m, 2H), 7.50-7.44 (m, 2H), 7.12 (t, J=8.8 Hz, 2H), 5.03 (dd, J=19.1, 14.2 Hz, 2H), 4.47 (s, 1H), 3.95 (s, 3H), 2.71 (s, 3H), 2.41 (br s, 4H), 2.27 (br s, 4H), 2.12 (s, 3H).

### Example 44

### Chiral analysis of N-[(3-cyano-2-methoxy-1-naphthyl)methyl]-2-(4-fluorophenyl)-N-methyl-2-(4-methylpiperazin-1-yl)acetamide

F-19 NMR spectra acquired in the presence of the chiral shift reagent 2,2,2-trifluoro-1-(9-anthryl)ethanol-d₁₁ were consistent with an enantiomeric excess of >98% for each isomer.

### Example 45

### N-[(3-cyano-2-methoxy-1-naphthyl)methyl]-2-(4-fluorophenyl)-N-methyl-2-piperazin-1-ylacetamide

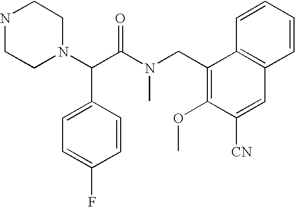

The title compound was prepared in a manner analogous to that described in Example 2 using chiral tert-butyl 4-[2-[[(3-cyano-2-methoxy-1-naphthyl)methyl](methyl)amino]-1-(4-fluorophenyl)-2-oxoethyl]piperazine-1-carboxylate. MS m/z 447.3 (M+H)⁺. ¹H NMR (300.132 MHz, DMSO) δ 8.60 (s, 1H), 8.01 (d, J=7.6 Hz, 1H), 7.90 (d, J=8.5 Hz, 1H), 7.60-7.53 (m, 2H), 7.51-7.44 (m, 2H), 7.12 (t, J=8.8 Hz, 2H), 5.03 (dd, J=24.3, 14.2 Hz, 2H), 4.44 (s, 1H), 3.95 (s, 3H), 2.72 (s, 3H), 2.72 (br s, 4H), 2.33 (br s, 4H).

### Example 46

### Chiral analysis of N-[(3-cyano-2-methoxy-1-naphthyl)methyl]-2-(4-fluorophenyl)-N-methyl-2-piperazin-1-ylacetamide

A 400 MHz proton NMR spectra with the addition of the chiral solvation reagent t-butylphenylphosphinothioic acid (TBPTA) were consistent with an enantiomeric excess of >99% for each isomer. There was no evidence in NMR spectra for the presence of the opposite enantiomer at levels above 0.5% of the total.

The requisite tert-butyl 4-[2-[[(3-cyano-2-methoxy-1-naphthyl)methyl](methyl)amino]-1-(4-fluorophenyl)-2-oxoethyl]piperazine-1-carboxylate was prepared in the following manner.

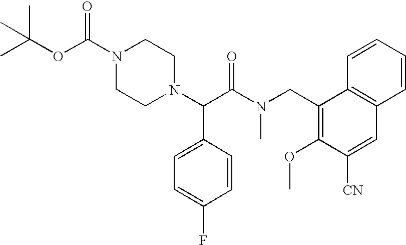

The title compound was prepared in a manner analogous to that described for tert-butyl 4-[2-[[(3-cyano-2-methoxy-1-naphthyl)methyl](methyl)amino]-1-(3-methoxyphenyl)-2-oxoethyl]piperazine-1-carboxylate. MS m/z 547.1 (M+H)⁺.¹H NMR (300.132 MHz, DMSO) δ 8.60 (s, 1H), 8.04-8.00 (m, 1H), 7.93-7.90 (m, 1H), 7.59-7.56 (m, 2H), 7.48-7.44 (m, 2H), 7.13 (t, J=8.8 Hz, 2H), 5.04 (s, 2H), 4.58 (s, 1H), 3.95 (s, 3H), 2.68 (s, 3H), 3.27 (br s, 4H), 2.40 (br s, 4H), 1.37 (s, 9H).

### Example 47

### Chiral Separation of tert-butyl 4-[2-[[(3-cyano-2-methoxy-1-naphthyl)methyl](methyl)amino]-1-(4-fluorophenyl)-2-oxoethyl]piperazine-1-carboxylate

Preparative Chromatography on tert-butyl 4-[2-[[(3-cyano-2-methoxy-1-naphthyl)methyl](methyl)amino]-1-(4-fluorophenyl)-2-oxoethyl]piperazine-1-carboxylate was carried out on a Chiralpak AD column (50×500 mm, 20 μm) using a mobile phase of 10% isopropanol and hexane at a flow rate of 100 mL/min with detection at 254 nm. A stock solution of the racemate was prepared at 1 g/18 mL in 0.4 mL DCM, 1.8 mL isopropanol and 15.8 mL of hexane. Sample injections were 9 mL of the stock solution (500 mg/injection). Chiral purity was assessed by analysis on a Chiralpak AD column (4.6×250 mm, 10 μm) using a mobile phase of 10% isopropanol and hexane at a flow rate of 1.0 mL/min with detection at 254 nm. Isomer 1: T_{R}=7.96 min; 89% ee. Isomer 2: T_{R}=10.82 min; 89% ee.

### Example 48

### N-[(3-cyano-2-methoxy-1-naphthyl)methyl]-2-(3-methoxyphenyl)-N-methyl-2-pyrrolidin-1-ylacetamide

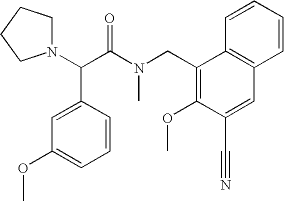

The title compound was prepared in a manner analogous to that described in Example 3. ¹H NMR (300.132 MHz, DMSO) δ 8.59 (s, 1H), 8.03-7.95 (m, 2H), 7.60-7.51 (m, 2H), 7.23-7.17 (m, 1H), 7.02 (s, 2H), 6.83-6.81 (m, 1H), 5.02 (dd, J=37.4, 13.8 Hz, 2H), 4.34 (s, 1H), 3.94 (s, 3H), 3.70 (s, 3H), 2.70 (s, 3H), 2.36 (br s, 4H), 1.67 (br s, 4H).

### Example 49

### Chiral Separation of N-[(3-cyano-2-methoxy-1-naphthyl)methyl]-2-(3-methoxyphenyl)-N-methyl-2-pyrrolidin-1-ylacetamide

Preparative Chromatography on N-[(3-cyano-2-methoxy-1-naphthyl)methyl]-2-(3-methoxyphenyl)-N-methyl-2-pyrrolidin-1-ylacetamide was carried out on a Chiralpak AD column (20×250 mm, 10 μ) using a mobile phase of 10% isopropanol and hexane at a flow rate of 20 mL/min with detection at 210 nm. A stock solution of the racemate was prepared at 400 mg/20 mL in 0.4 mL DCM, 2.0 mL isopropanol and 17.6 mL of hexane. Sample injections were 5 mL of the stock solution (100 mg/injection). Chiral purity was assessed by analysis on a Chiralpak AD column (4.6×250 mm, 10 μm) using a mobile phase of 10% isopropanol and hexane at a flow rate of 1.0 mL/min with detection at 210 nm. Isomer 1: T_{R}=9.03 min; >99% ee. Isomer 2: T_{R}=12.45 min; >99% ee.

### Example 50

### N-{2-[3,5-bis(trifluoromethyl)phenyl]ethyl}-2-(4-fluorophenyl)-N-methyl-2-(3-morpholin-4-ylazetidin-1-yl)acetamide

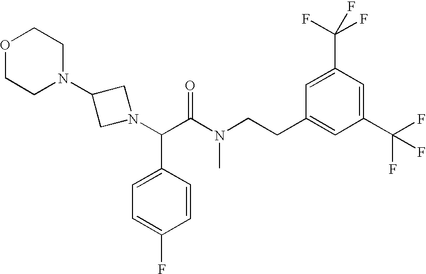

2-(3-aminoazetidin-1-yl)-N-{2-[3,5-bis(trifluoromethyl)phenyl]ethyl}-2-(4-fluorophenyl)-N-methylacetamide (136 mg, 0.285 mmol) was dissolved in ACN (10 mL) and Na₂CO₃ (600 mg 5.7 mmol) was added followed by addition of diethylene glycol di-p-tosylate (130 mg, 0.313 mmol). The mixture was refluxed for two days and reaction cooled to room temperature. H₂O (10 mL) and EtOAc (10 mL) were added and layers separated. The organic phase was dried over Na₂SO₄, filtered through a pad of diatomaceous earth and the volatiles removed under reduce pressure. Chromatography of the residue on SiO₂ (0-10% MeOH:DCM) afforded the title compound (42 mg, 27%). MS m/z 548.2 (M+H)⁺. ¹H NMR (300.132 MHz, CDCl3) δ 7.70 (s, 1H), 7.60 (s, 2H), 7.32-7.28 (m, 2H), 7.09-6.98 (m, 2H), 4.17 (s, 1H), 3.69 (br s, 4H), 3.64-3.49 (m, 4H), 3.22-3.18 (m, 1H), 3.04-2.83 (m, 4H), 2.79 (s, 3H), 2.26 (br s, 4H).

### Example 51

### N-[(3-cyano-2-methoxy-1-naphthyl)methyl]-2-(4-fluorophenyl)-N-methyl-2-(methylamino)acetamide

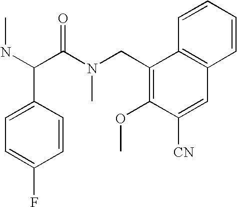

2-[Allyl(methyl)amino]-N-[(3-cyano-2-methoxy-1-naphthyl)methyl]-2-(4-fluorophenyl)-N-methylacetamide (1.48 g, 3.43 mmol) was dissolved in dry THF (10 mL) and the mixture purged with nitrogen for 15 min. In a separate flask, Pd(dba)₂ (197 mg, 0.343 mmol) and 1,4-bis(diphenylphosphino)butane (146 mg, 0.343 mmol) were premixed in purged THF (5 mL) for 20 min. This was added to the amine in one portion followed by rapid drop wise addition of thiosalicylic acid (635 mg, 4.11 mmol) dissolved in THF (2 mL). The mixture was stirred for 30 min. Reaction mixture diluted with EtOAc (50 mL) and washed with Na₂CO₃ (25 mL), H₂O (25 mL), and brine (20 mL). The organic phase was dried over Na₂SO₄, filtered through a pad of diatomaceous earth and the volatiles removed under reduce pressure. Chromatography of the residue on SiO₂ (0-5% MeOH:DCM) afforded the title compound (901 mg, 67%). MS m/z 392.1 (+H)⁺. ¹H NMR (300.132 MHz, DMSO) δ 8.61 (s, 1H), 8.04-7.97 (m, 2H), 7.61-7.58 (m, 2H), 7.41-7.36 (m, 2H), 7.11 (t, J=8.8 Hz, 2H), 5.08 (s, 2H), 4.63 (s, 1H), 3.96 (s, 3H), 2.63 (s, 3H), 2.21 (s, 3H).

The requisite 2-[allyl(methyl)amino]-N-[(3-cyano-2-methoxy-1-naphthyl)methyl]-2-(4-fluorophenyl)-N-methylacetamide was prepared in a manner analogous to that described in Example 3:

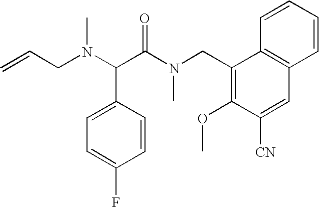

### Example 52

### 2-(4-fluorophenyl)-N-{[2-methoxy-3-(trifluoromethyl)-5 6,7,8-tetrahydronaphthalen-1-yl]methyl}-N-methyl-2-piperazin-1-ylacetamide

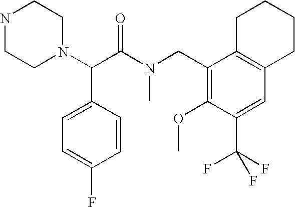

The title compound was prepared in a manner analogous to that described in Example 2. The requisite 1-[2-methoxy-3-(trifluoromethyl)-5,6,7,8-tetrahydronaphthalen-1-yl]-N-methylmethanamine was synthesized in a manner analogous to that described for 3-methoxy-4-[(methylamino)methyl]-2-naphthonitrile. MS m/z 274.2 (M+H)⁺.

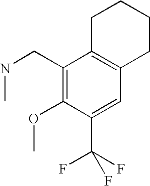

The requisite 1-(bromomethyl)-3-(trifluoromethyl)-5,6,7,8-tetrahydronaphthalen-2-yl methyl ether was synthesized using the following method.

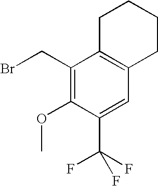

[2-Methoxy-3-(trifluoromethyl)-5,6,7,8-tetrahydronaphthalen-1-yl]methanol (421 mg, 1.61 mmol) was dissolved in THF (20 mL) and placed an in ice water bath. CBr₄ (644 mg, 1.93 mmol) was added followed by addition of PPh₃ (509 mg, 1.93 mmol) in portions. Reaction was allowed to warm to room temperature over night. The solids were filtered off through a pad of diatomaceous earth and the filtrate was diluted with EtOAc (25 mL), washed with H₂O (10 mL) and brine (5 mL). The organic phase was dried over Na₂SO₄, filtered through a pad of diatomaceous earth and the volatiles removed under reduce pressure. Chromatography of the residue on SiO₂ (0-20% EA:HEX)) afforded the title compound (300 mg, 60%). ¹H NMR (300.132 MHz, CDCl3) δ 7.28 (s, 1H), 4.60 (s, 2H), 3.98 (s, 3H), 2.86 (t, J=6.1 Hz, 2H), 2.77 (t, J=6.1 Hz, 2H), 1.92-1.75 (m, 4H).

The requisite [2-methoxy-3-(trifluoromethyl)-5,6,7,8-tetrahydronaphthalen-1-yl]methanol was synthesized using the following method.

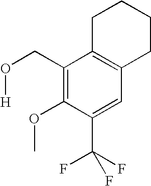

2-Methoxy-3 trifluoromethyl-5,6,7,8-tetrahydro-naphthalene-1-carboxylic acid methyl ester (519 mg, 1.80 mmol) was dissolved in ether (25 mL) and cooled in an ice water bath. LAH (136 mg, 3.6 mmol) was slowly added under nitrogen in portions and reaction stirred at 0° C. for 2 h. Reaction was quenched with Na₂SO₄ (sat, aq, 4 mL) and stirred for 20 min. Solid Na₂SO₄ (300 mg) was added and mixture filtered through a pad of diatomaceous earth. The filtrate was diluted with EtOAc (25 mL), washed with H₂O (10 mL) and brine (5 mL). The organic phase was dried over Na₂SO₄, filtered through a pad of diatomaceous earth and the volatiles removed under reduce pressure. Chromatography of the residue on SiO₂ (0-50% EA:HEX)) afforded the title compound (421 mg, 90%). MS m/z 243.1 (M+H-OH)⁺. ¹H NMR (300.132 MHz, DMSO) δ 7.28 (s, 1H), 4.95 (s, 1H), 4.53 (s, 2H), 3.80 (s, 3H), 2.92-2.88 (m, 2H), 2.76-2.72 (m, 2H), 1.75-1.70 (m, 4H).

The requisite methyl 2-methoxy-3-(trifluoromethyl)-5,6,7,8-tetrahydronaphthalene-1-carboxylate was prepared by the following method.

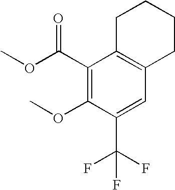

A suspension containing 3-bromo-2-methoxy-5,6,7,8-tetrahydro-naphthalene-1-carboxylic acid methyl ester (2.0 g, 6.69 mmol), potassium trifluoroacetate (1.96 g, 10 mmol), copper (1) iodide (2.67 g, 14 mmol), and dry DMF (50 mL) was heated to reflux for 2 h. Temperature was reduced to 115° C. and held for 48 h. The reaction was cooled to room temp and poured into dilute aqueous HCl (2N, 150 mL). This slurry was vacuum filtered through a medium sintered glass filter using ethyl acetate washes (6×30 mL). The combined filtrates were washed with 1N aq. HCl (100 mL), saturated sodium thiosulfate (100 mL), and then sat. aq. brine (100 mL). The organic layer was dried over magnesium sulfate, filtered, and concentrated. The residue was purified by flash silica gel chromatography (3:1 hexane/methylene chloride then 3:2 hexane/methylene chloride) to give the title compound (0.67 g, 35% yield) as a tan oil. MS m/z 289 (M+H). ¹H NMR (300.132 MHz, CDCl₃) δ 7.30 (s, 1H), 3.92 (s, 3H), 3.84 (s, 3H), 2.70 (m, 4H), 1.78 (m, 4H). ¹⁹F NMR (CDCl₃) δ-60.44 (s).

### Example 53

### N-[(3-cyano-2-methoxy-1-naphthyl)methyl]-2-(4-fluorophenyl)-N-methyl-2-[(1-methylpyrrolidin-3-yl)amino]acetamide

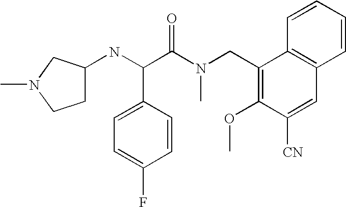

N-[2-[[(3-Cyano-2-methoxy-1-naphthyl)methyl](methyl)amino]-1-(4-fluorophenyl)-2-oxoethyl]-2,2,2-trifluoro-N-(1-methylpyrrolidin-3-yl)acetamide (33 mg, 0.059 mmol) was dissolved in MeOH (5 mL) and K₂CO₃ (82 mg, 0.59 mmol) dissolved in water (2 mL) was added and reaction mixture stirred for 2 h. The reaction was extracted with DCM (10 mL) and the organic phase was dried over Na₂SO₄, filtered through a pad of diatomaceous earth and the volatiles removed under reduce pressure. Chromatography of the residue on SiO₂ (0-5% 2 M NH3 MeOH:DCM) afforded the title compound (17 mg, 62%). MS m/z 461.3 (M+H)⁺. ¹H NMR (300.132 MHz, CDCl3) δ 8.20 (s, 2H), 7.84 (d, J=7.9 Hz, 1H), 7.61-7.51 (m, 2H), 7.38-7.34 (m, 2H), 6.98 (t, J=8.2 Hz, 2H), 5.34 (dd, J=16.8, 14.1 Hz, 1H), 5.03 (t, J=13.5 Hz, 1H), 4.61 (d, J=20.4 Hz, 1H), 4.03 (s, 3H), 3.51-3.26 (m, 1H), 3.08-2.76 (m, 4H), 2.64 (d, J=4.7 Hz, 3H), 2.35 (d, J=4.8 Hz, 3H), 1.89-1.65 (m, 2H).

The requisite N-[2-[[(3-cyano-2-methoxy-1-naphthyl)methyl](methyl)amino]-1-(4-fluorophenyl)-2-oxoethyl]-2,2,2-trifluoro-N-(1-methylpyrrolidin-3-yl)acetamide was prepared in a manner analogous to that described in Example 1 using N-[2-[[(3-cyano-2-methoxy-1-naphthyl)methyl](methyl)amino]-1-(4-fluorophenyl)-2-oxoethyl]-2,2,2-trifluoro-N-pyrrolidin-3-ylacetamide. MS m/z 557.2 (M+H)⁺.

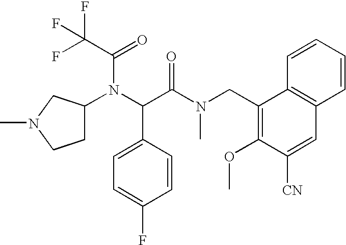

The requisite N-[2-[[(3-cyano-2-methoxy-1-naphthyl)methyl](methyl)amino]-1-(4-fluorophenyl)-2-oxoethyl]-2,2,2-trifluoro-N-pyrrolidin-3-ylacetamide was prepared in a manner analogous to that described in Example 2 using tert-butyl 3-[[2-[[(3-cyano-2-methoxy-1-naphthyl)methyl]-(methyl)amino]-1-(4-fluorophenyl)-2-oxoethyl]-(trifluoroacetyl)amino]pyrrolidine-1-carboxylate. MS m/z 543.0 (M+H)⁺.

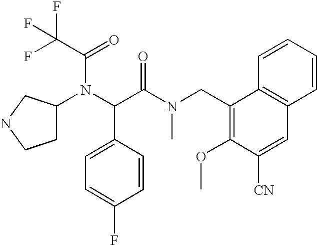

The requisite tert-butyl 3-[[2-[[(3-cyano-2-methoxy-1-naphthyl)methyl](methyl)amino]-1-(4-fluorophenyl)-2-oxoethyl](trifluoroacetyl)amino]pyrrolidine-1-carboxylate was prepared in a manner analogous to that described within Example 2 using [[1-(tert-butoxycarbonyl)pyrrolidin-3-yl](trifluoroacetyl)amino](4-fluorophenyl)acetic acid. MS m/z 542.9 (M+H-BOC).

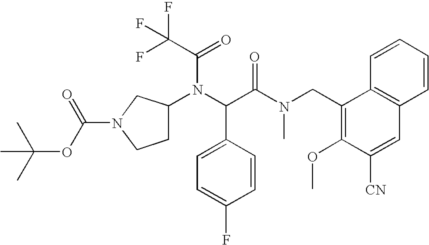

The requisite [[1-(tert-butoxycarbonyl)pyrrolidin-3-yl](trifluoroacetyl)amino](4-fluorophenyl)acetic acid was synthesized using the following method.

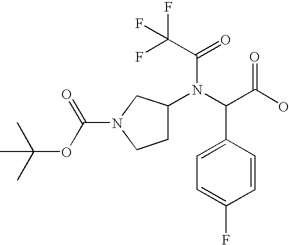

{[1-(tert-Butoxycarbonyl)pyrrolidin-3-yl]amino} (4-fluorophenyl)acetic acid (826 mg, 2.44 mmol) was dissolved in DCM (50 mL) and cooled in an ice water bath. Et₃N (1.0 mL, 7.32 mmol) was added and the reaction was bubbled with trifluoroacetyl chloride for 30 sec. After 2 h, the volatiles were removed under reduced pressure and crude was taken up in DCM and stirred with water for 20 min. The mixture was acidified with 1N HCl, the organic layer separated and the volatiles were removed under reduced pressure. The product was triturated with Et₂O to afforded the title compound as a yellowish solid (622 mg, 59%). MS m/z 335, (M+H-BOC). ¹H NMR (300.132 MHz, DMSO) δ 7.44-7.34 (m, 2H), 7.27-7.13 (m, 2H), 5.68-5.49 (m, 1H), 3.56-3.12 (m, 7H), 1.40-1.28 (m, 9H).

The requisite {[1-(tert-butoxycarbonyl)pyrrolidin-3-yl]amino}(4-fluorophenyl)acetic acid was prepared in a manner analogous to that described for [4-(tert-butoxycarbonyl)piperazin-1-yl](3-methoxyphenyl)acetic acid. MS m/z 339.2 (M+H)⁺, 283.1 (M+H-t-butyl).

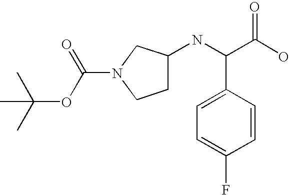

### Example 54

### N-[(3-cyano-2-methoxy-1-naphthyl)methyl]-2-(4-fluorophenyl)-N-methyl-2-[methyl(pyrrolidin-3-yl)amino]acetamide

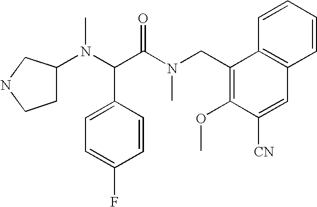

The title compound was prepared in a manner analogous to that described in Example 2 using tert-butyl 3-[[2-[[(3-cyano-2-methoxy-1-naphthyl)methyl]-(methyl)amino]-1-(4-fluorophenyl)-2-oxoethyl](methyl)amino]pyrrolidine-1-carboxylate. MS m/z 461.3 (M+H)⁺.

The requisite tert-butyl 3-[[2-[[(3-cyano-2-methoxy-1-naphthyl)methyl]-(methyl)amino]-1-(4-fluorophenyl)-2-oxoethyl](methyl)amino]pyrrolidine-1-carboxylate was synthesized using the following method.

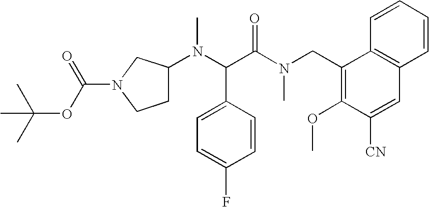

tert-Butyl 3-{[2-[[(3-cyano-2-methoxy-1-naphthyl)methyl](methyl)amino]-1-(4-fluorophenyl)-2-oxoethyl]amino}pyrrolidine-1-carboxylate (60 mg, 0.109 mmol) was dissolved in ACN (2 mL) and paraformaldehyde (6 mg, 0.218 mmol ) and 1 drop of CH₃CO₂H was added under nitrogen. After 2 h, NaCNBH₃ (10 mg, 0.163 mmol) was added and the reaction was stirred overnight. EtOAc (10 mL) and H20 (10 mL) were added and the resulting layers separated. The organic phase was dried over Na₂SO₄, filtered through a pad of diatomaceous earth and the volatiles removed under reduce pressure. Chromatography of the residue on SiO₂ (0-75% EA:HEX) afforded the title compound (20 mg, 33%). MS m/z 561.2 (M+H)⁺. ¹H NMR (300.132 MHz, DMSO) δ 8.63 (s, 1H), 8.14-8.03 (m, 2H), 7.70-7.59 (m, 2H), 7.44-7.39 (m, 2H), 7.16 (t, J=8.7 Hz, 2H), 5.34-5.26 (m, 1H), 4.93-4.77 (m, 2H), 3.98 (s, 3H), 3.43-3.00 (m, 6H), 2.27 (s, 3H), 2.57 (s, 3H), 1.92-1.72 (m, 1H), 1.38 (s, 9H).

The requisite tert-butyl 3-{[2-[[(3-cyano-2-methoxy-1-naphthyl)methyl](methyl)amino]-1-(4-fluorophenyl)-2-oxoethyl]amino}pyrrolidine-1-carboxylate was prepared in a manner analogous to that described for N-[(3-cyano-2-methoxy-1-naphthyl)methyl]-2-(4-fluorophenyl)-N-methyl-2-[(1-methylpyrrolidin-3-yl)amino]acetamide.

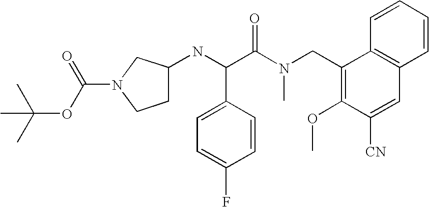

### Examples 55-120:

The following compounds were prepared in a manner analogous to those described in Examples 1-54:

| Ex. | | | | MS m/z |
|---|---|---|---|---|
| No. | Structure | Chemical name | MW | (M + H)+ |
| 55 | 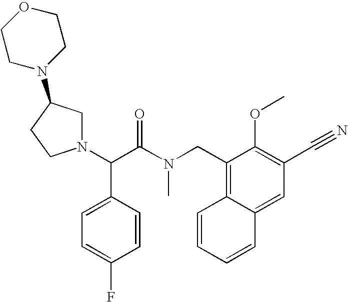 | N-[(3-cyano-2-methoxy-1- naphthyl)methyl]-2-(4-fluorophenyl)- N-methyl-2-[(3R)-3-morpholin-4- ylpyrrolidin-1-yl]acetamide | 516.61 | 517.3 |
| 56 | 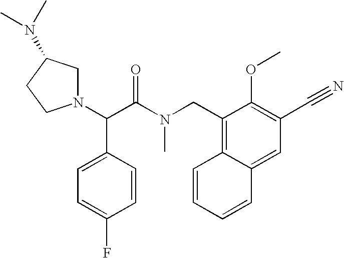 | N-[(3-cyano-2-methoxy-1- naphthyl)methyl]-2-[(3S)-3- (dimethylamino)pyrrolidin-1-yl]-2-(4- fluorophenyl)-N-methylacetamide | 474.58 | 475.2 |
| 57 | 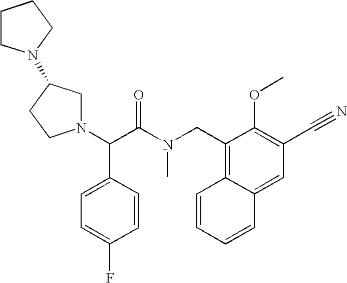 | 2-[(3′S)-1,3′-bipyrrolidin-1′-yl]-N-[(3- cyano-2-methoxy-1-naphthyl)methyl]- 2-(4-fluorophenyl)-N- methylacetamide | 500.62 | 501.2 |
| 58 | 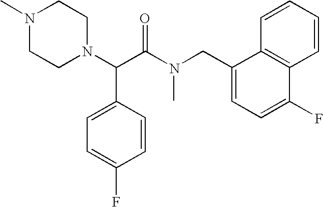 | N-[(4-fluoro-1-naphthyl)methyl]-2-(4- fluorophenyl)-N-methyl-2-(4- methylpiperazin-1-yl)acetamide | 423.51 | 424.2 |
| 59 | 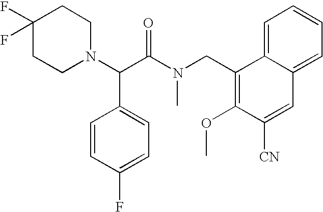 | N-[(3-cyano-2-methoxy-1- naphthyl)methyl]-2-(4,4- difluoropiperidin-1-yl)-2-(4- fluorophenyl)-N-methylacetamide | 481.52 | 482.3 |
| 60 | 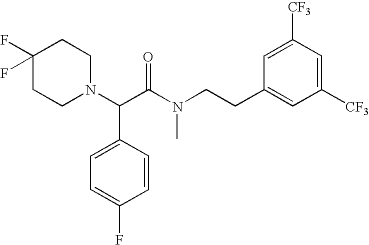 | N-{2-[3,5- bis(trifluoromethyl)phenyl]ethyl}-2- (4,4-difluoropiperidin-1-yl)-2-(4- fluorophenyl)-N-methylacetamide | 526.44 | 527.1 |
| 61 | 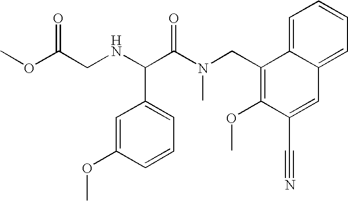 | methyl {[2-[[(3-cyano-2-methoxy-1- naphthyl)methyl](methyl)amino]-1-(3- methoxyphenyl)-2- oxoethyl]amino}acetate | 461.52 | 462.2 |
| 62 | 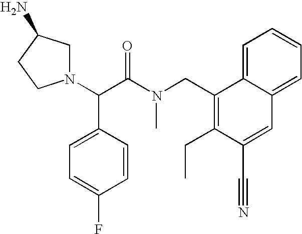 | 2-[(3R)-3-aminopyrrolidin-1-yl]-N- [(3-cyano-2-ethyl-1-naphthyl)methyl]- 2-(4-fluorophenyl)-N- methylacetamide | 444.55 | 445.2 |
| 63 | 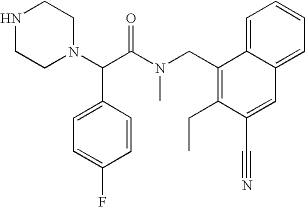 | N-[(3-cyano-2-ethyl-1- naphthyl)methyl]-2-(4-fluorophenyl)- N-methyl-2-piperazin-1-ylacetamide | 444.55 | 445.2 |
| 64 | 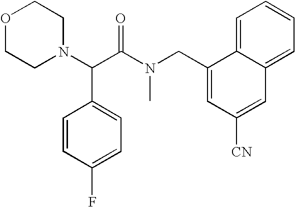 | N-[(3-cyano-1-naphthyl)methyl]-2-(4- fluorophenyl)-N-methyl-2-morpholin- 4-ylacetamide | 417.48 | 418.3 |
| 65 | 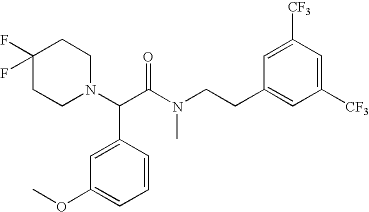 | N-{2-[3,5- bis(trifluoromethyl)phenyl]ethyl}-2- (4,4-difluoropiperidin-1-yl)-2-(3- methoxyphenyl)-N-methylacetamide | 538.48 | 539.1 |
| 66 | 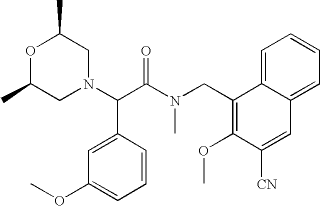 | N-[(3-cyano-2-methoxy-1- naphthyl)methyl]-2-[(2R,6S)-2,6- dimethylmorpholin-4-yl]-2-(3- methoxyphenyl)-N-methylacetamide | 487.60 | 488.3 |
| 67 | 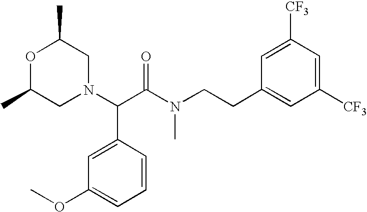 | N-{2-[3,5- bis(trifluoromethyl)phenyl]ethyl}-2- [(2R,6S)-2,6-dimethylmorpholin-4- yl]-2-(3-methoxyphenyl)-N- methylacetamide | 532.52 | 533.3 |
| 68 | 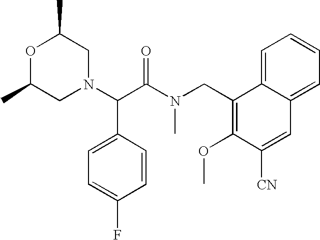 | N-[(3-cyano-2-methoxy-1- naphthyl)methyl]-2-[(2R,6S)-2,6- dimethylmorpholin-4-yl]-2-(4- fluorophenyl)-N-methylacetamide | 475.56 | 476.2 |
| 69 | 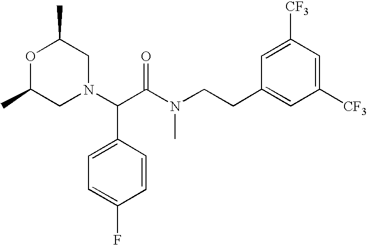 | N-{2-[3,5- bis(trifluoromethyl)phenyl]ethyl}-2- [(2R,6S)-2,6-dimethylmorpholin-4- yl]-2-(4-fluorophenyl)-N- methylacetamide | 520.49 | 521.3 |
| 70 | 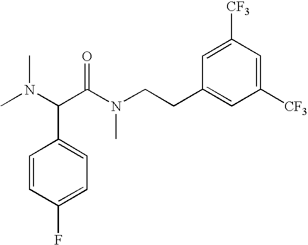 | N-{2-[3,5- bis(trifluoromethyl)phenyl]ethyl}-2- (dimethylamino)-2-(4-fluorophenyl)- N-methylacetamide | 450.40 | 451.2 |
| 71 | 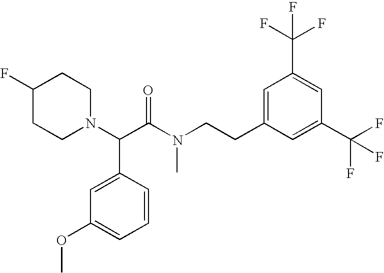 | N-{2-[3,5- bis(trifluoromethyl)phenyl]ethyl}-2- (4-fluoropiperidin-1-yl)-2-(3- methoxyphenyl)-N-methylacetamide | 520.49 | 521.2 |
| 72 | 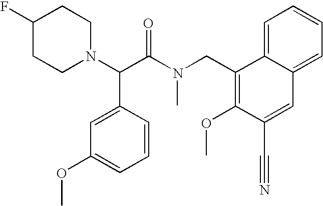 | N-[(3-cyano-2-methoxy-1- naphthyl)methyl]-2-(4-fluoropiperidin- 1-yl)-2-(3-methoxyphenyl)-N- methylacetamide | 475.56 | 476.3 |
| 73 | 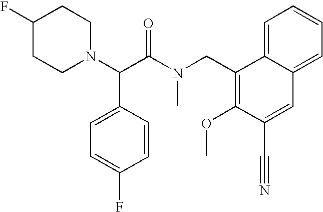 | N-[(3-cyano-2-methoxy-1- naphthyl)methyl]-2-(4-fluorophenyl)- 2-(4-fluoropiperidin-1-yl)-N- methylacetamide | 463.52 | 464.3 |
| 74 | 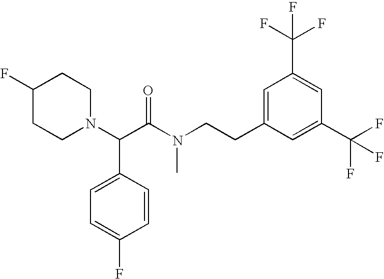 | N-{2-[3,5- bis(trifluoromethyl)phenyl]ethyl}-2- (4-fluorophenyl)-2-(4-fluoropiperidin- 1-yl)-N-methylacetamide | 508.45 | 509.2 |
| 75 | 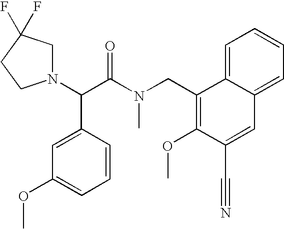 | N-[(3-cyano-2-methoxy-1- naphthyl)methyl]-2-(3,3- difluoropyrrolidin-1-yl)-2-(3- methoxyphenyl)-N-methylacetamide | 479.52 | 480.2 |
| 76 | 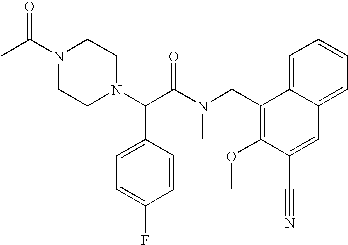 | 2-(4-acetylpiperazin-1-yl)-N-[(3- cyano-2-methoxy-1-naphthyl) methyl]- 2-(4-fluorophenyl)-N- methylacetamide | 488.56 | 489.3 |
| 77 | 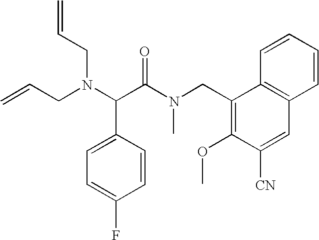 | N-[(3-cyano-2-methoxy-1- naphthyl)methyl]-2-(diallylamino)-2- (4-fluorophenyl)-N-methylacetamide | 457.54 | 458.2 |
| 78 | 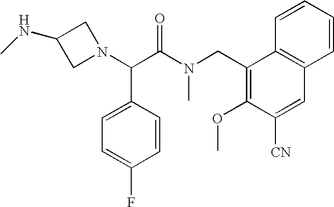 | N-[(3-cyano-2-methoxy-1- naphthyl)methyl]-2-(4-fluorophenyl)- N-methyl-2-[3-(methylamino)azetidin- 1-yl]acetamide | 446.53 | 447.3 |
| 79 | 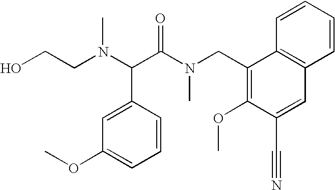 | N-[(3-cyano-2-methoxy-1- naphthyl)methyl]-2-[(2- hydroxyethyl)(methyl)amino]-2-(3- methoxyphenyl)-N-methylacetamide | 447.5 | 448.3 |
| 80 | 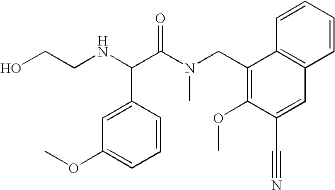 | N-[(3-cyano-2-methoxy-1- naphthyl)methyl]-2-[(2- hydroxyethyl)amino]-2-(3- methoxyphenyl)-N-methylacetamide | 443.5 | 434.3 |
| 81 | 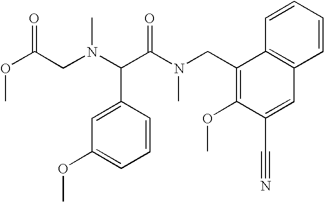 | methyl N-[2-[[(3-cyano-2-methoxy-1- naphthyl)methyl](methyl)amino]-1-(3- methoxyphenyl)-2-oxoethyl]-N- methylglycinate | 475.5 | 476.2 |
| 82 | 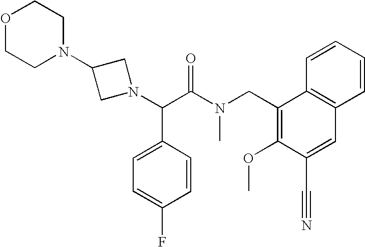 | N-[(3-cyano-2-methoxy-1- naphthyl)methyl]-2-(4-fluorophenyl)- N-methyl-2-(3-morpholin-4- ylazetidin-1-yl)acetamide | 502.50 | 503.3 |
| 83 | 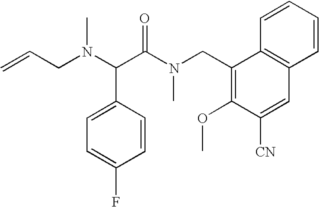 | 2-[allyl(methyl)amino]-N-[(3-cyano-2- methoxy-1-naphthyl)methyl]-2-(4- fluorophenyl)-N-methylacetamide | 431.50 | 432.2 |
| 84 | 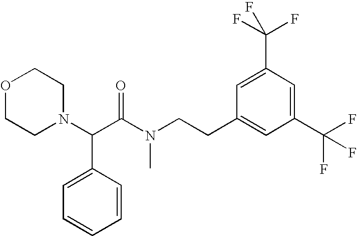 | N-{2-[3,5- bis(trifluoromethyl)phenyl]ethyl}-N- methyl-2-morpholin-4-yl-2- phenylacetamide | 474.40 | 475.2 |
| 85 | 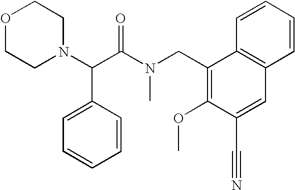 | N-[(3-cyano-2-methoxy-1- naphthyl)methyl]-N-methyl-2- morpholin-4-yl-2-phenylacetamide | 429.50 | 430.2 |
| 86 | 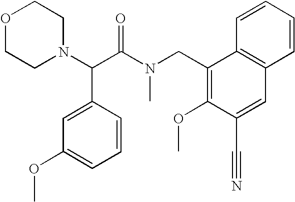 | N-[(3-cyano-2-methoxy-1- naphthyl)methyl]-2-(3- methoxyphenyl)-N-methyl-2- morpholin-4-ylacetamide | 459.50 | 460.2 |
| 87 | 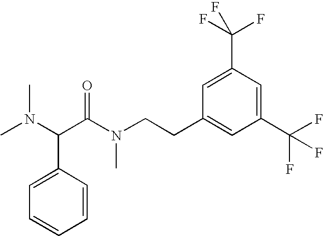 | N-{2-[3,5- bis(trifluoromethyl)phenyl]ethyl}-2- (dimethylamino)-N-methyl-2- phenylacetamide | 432.40 | 433.1 |
| 88 | 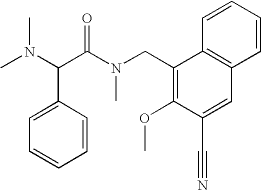 | N-[(3-cyano-2-methoxy-1- naphthyl)methyl]-2-(dimethylamino)- N-methyl-2-phenylacetamide | 387.40 | 388.1 |
| 89 | 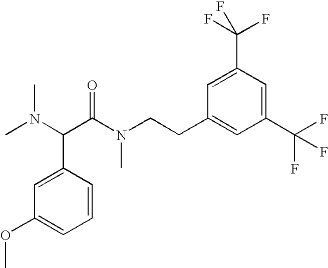 | N-{2-[3,5- bis(trifluoromethyl)phenyl]ethyl}-2- (dimethylamino)-2-(3- methoxyphenyl)-N-methylacetamide | 462.40 | 463.1 |
| 90 | 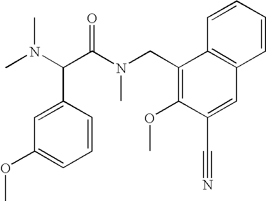 | N-[(3-cyano-2-methoxy-1- naphthyl)methyl]-2-(dimethylamino)- 2-(3-methoxyphenyl)-N- methylacetamide | 417.50 | 418.1 |
| 91 | 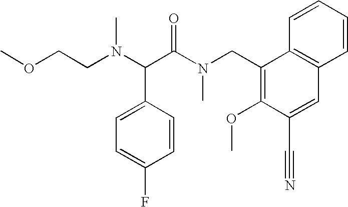 | N-[(3-cyano-2-methoxy-1- naphthyl)methyl]-2-(4-fluorophenyl)- 2-[(2-methoxyethyl)(methyl)amino]- N-methylacetamide | 449.53 | 450.3 |
| 92 | 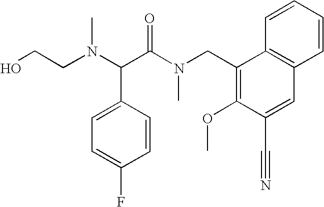 | N-[(3-cyano-2-methoxy-1- naphthyl)methyl]-2-(4-fluorophenyl)- 2-[(2-hydroxyethyl)(methyl)amino]-N- methylacetamide | 435.50 | 436.3 |
| 93 | 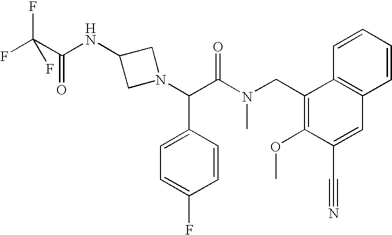 | N-{1-[2-[[(3-cyano-2-methoxy-1- naphthyl)methyl](methyl)amino]-1-(4- fluorophenyl)-2-oxoethyl]azetidin-3- yl}-2,2,2-trifluoroacetamide | 528.51 | 529.2 |
| 94 | 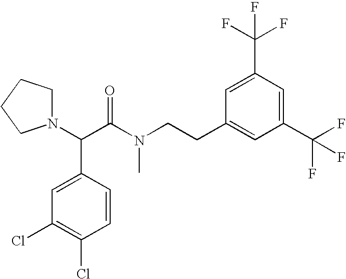 | N-{2-[3,5- bis(trifluoromethyl)phenyl]ethyl}-2- (3,4-dichlorophenyl)-N-methyl-2- pyrrolidin-1-ylacetamide | 527.33 | 527.1 |
| 95 | 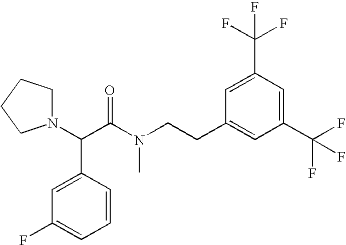 | N-{2-[3,5- bis(trifluoromethyl)phenyl]ethyl}-2- (3-fluorophenyl)-N-methyl-2- pyrrolidin-1-ylacetamide | 476.43 | 477.3 |
| 96 | 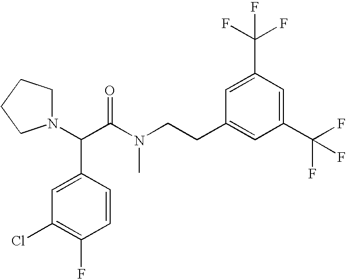 | N-{2-[3,5- bis(trifluoromethyl)phenyl]ethyl}-2- (3-chloro-4-fluorophenyl)-N-methyl- 2-pyrrolidin-1-ylacetamide | 510.88 | 511.1 |
| 97 | 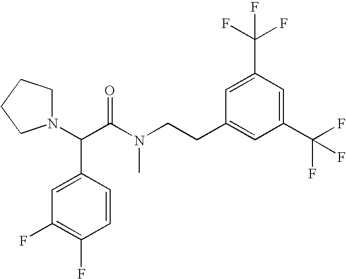 | N-{2-[3,5- bis(trifluoromethyl)phenyl]ethyl}-2- (3,4-difluorophenyl)-N-methyl-2- pyrrolidin-1-ylacetamide | 494.42 | 495.1 |
| 98 | 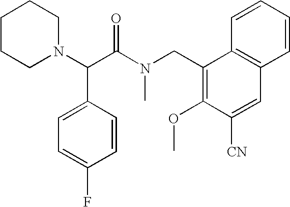 | N-[(3-cyano-2-methoxy-1- naphthyl)methyl]-2-(4-fluorophenyl)- N-methyl-2-piperidin-1-ylacetamide | 445.54 | 446.4 |
| 99 | 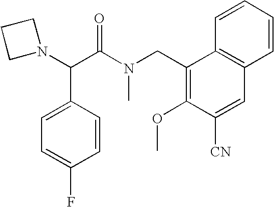 | 2-azetidin-1-yl-N-[(3-cyano-2- methoxy-1-naphthyl)methyl]-2-(4- fluorophenyl)-N-methylacetamide | 417.48 | 418.3 |
| 100 | 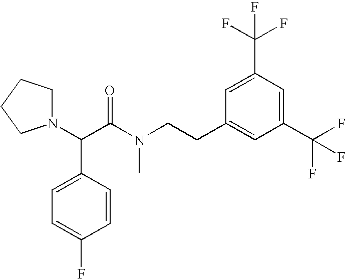 | N-{2-[3,5- bis(trifluoromethyl)phenyl]ethyl}-2- (4-fluorophenyl)-N-methyl-2- pyrrolidin-1-ylacetamide | 476.43 | 477.4 |
| 101 | 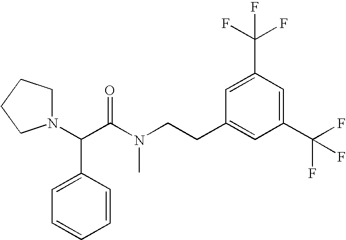 | N-{2-[3,5- bis(trifluoromethyl)phenyl]ethyl}-N- methyl-2-phenyl-2-pyrrolidin-1- ylacetamide | 458.44 | 459.4 |
| 102 | 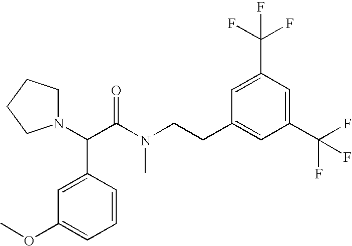 | N-{2-[3,5- bis(trifluoromethyl)phenyl]ethyl}-2- (3-methoxyphenyl)-N-methyl-2- pyrrolidin-1-ylacetamide | 488.47 | 489.1 |
| 103 | 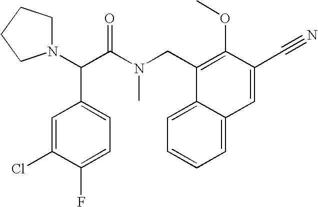 | 2-(3-chloro-4-fluorophenyl)-N-[(3- cyano-2-methoxy-1-naphthyl)methyl]- N-methyl-2-pyrrolidin-1-ylacetamide | 465.95 | 466.1 |
| 104 | 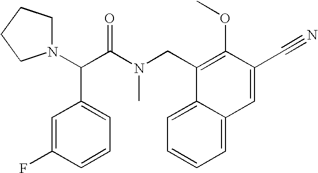 | N-[(3-cyano-2-methoxy-1- naphthyl)methyl]-2-(3-fluorophenyl)- N-methyl-2-pyrrolidin-1-ylacetamide | 431.51 | 432.1 |
| 105 | 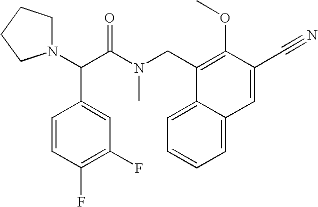 | N-[(3-cyano-2-methoxy-1- naphthyl)methyl]-2-(3,4- difluorophenyl)-N-methyl-2- pyrrolidin-1-ylacetamide | 449.50 | 450.0 |
| 106 | 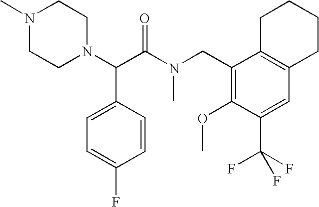 | 2-(4-fluorophenyl)-N-{[2-methoxy-3- (trifluoromethyl)-5,6,7,8- tetrahydronaphthalen-1-yl]methyl}-N- methyl-2-(4-methylpiperazin-1- yl)acetamide | 507.57 | 508.1 |
| 107 | 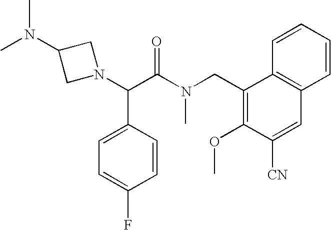 | N-[(3-cyano-2-methoxy-1- naphthyl)methyl]-2-[3- (dimethylamino)azetidin-1-yl]-2-(4- fluorophenyl)-N-methylacetamide | 460.56 | 461.1 |
| 108 | 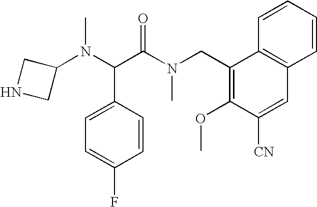 | 2-[azetidin-3-yl(methyl)amino]-N-[(3- cyano-2-methoxy-1-naphthyl)methyl]- 2-(4-fluorophenyl)-N- methylacetamide | 446.53 | 447.3 |
| 109 | 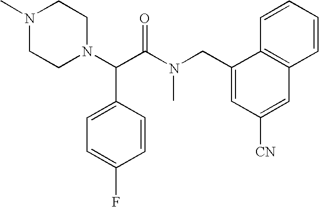 | N-[(3-cyano-1-naphthyl)methyl]-2-(4- fluorophenyl)-N-methyl-2-(4- methylpiperazin-1-yl)acetamide | 430.53 | 431.1 |
| 110 | 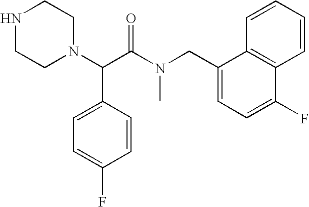 | N-[(4-fluoro-1-naphthyl)methyl]-2-(4- fluorophenyl)-N-methyl-2-piperazin- 1-ylacetamide | 409.48 | 410.1 |
| 111 | 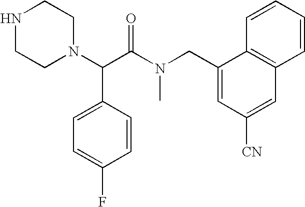 | N-[(3-cyano-1-naphthyl)methyl]-2-(4- fluorophenyl)-N-methyl-2-piperazin- 1-ylacetamide | 416.50 | 417.1 |
| 112 | 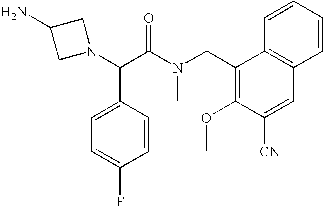 | 2-(3-aminoazetidin-1-yl)-N-[(3-cyano- 2-methoxy-1-naphthyl)methyl]-2-(4- fluorophenyl)-N-methylacetamide | 432.50 | 433.2 |
| 113 | 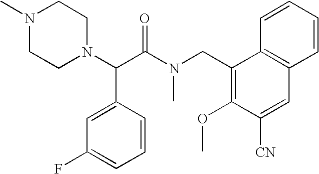 | N-[(3-cyano-2-methoxy-1- naphthyl)methyl]-2-(3-fluorophenyl)- N-methyl-2-(4-methylpiperazin-1- yl)acetamide | 460.56 | 461.2 |
| 114 | 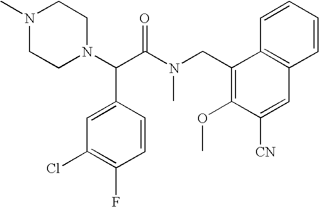 | 2-(3-chloro-4-fluorophenyl)-N-[(3- cyano-2-methoxy-1-naphthyl)methyl]- N-methyl-2-(4-methylpiperazin-1- yl)acetamide | 495.00 | 495.2 |
| 115 | 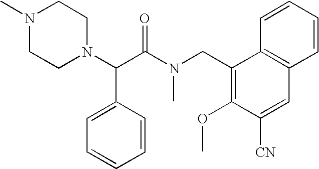 | N-[(3-cyano-2-methoxy-1- naphthyl)methyl]-N-methyl-2-(4- methylpiperazin-1-yl)-2- phenylacetamide | 442.57 | 443.2 |
| 116 | 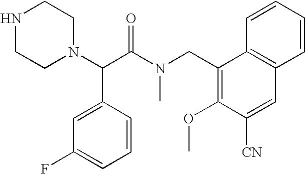 | N-[(3-cyano-2-methoxy-1- naphthyl)methyl]-2-(3-fluorophenyl)- N-methyl-2-piperazin-1-ylacetamide | 446.53 | 447.3 |
| 117 | 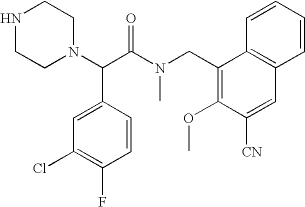 | 2-(3-chloro-4-fluorophenyl)-N-[(3- cyano-2-methoxy-1-naphthyl)methyl]- N-methyl-2-piperazin-1-ylacetamide | 480.97 | 481.2 |
| 118 | 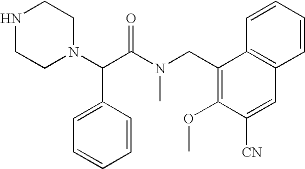 | N-[(3-cyano-2-methoxy-1- naphthyl)methyl]-N-methyl-2-phenyl- 2-piperazin-1-ylacetamide | 428.54 | 429.3 |
| 119 | 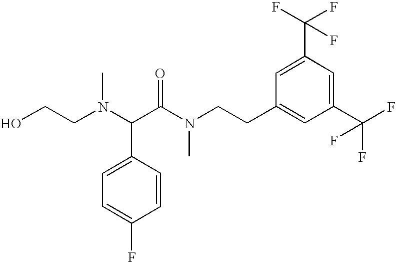 | N-{2-[3,5- bis(trifluoromethyl)phenyl]ethyl}-2- (4-fluorophenyl)-2-[(2- hydroxyethyl)(methyl)amino]-N- methylacetamide | 480.43 | 481.2 |
| 120 | 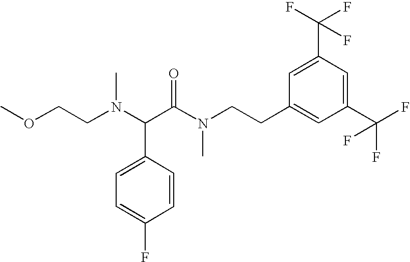 | N-{2-[3,5- bis(trifluoromethyl)phenyl]ethyl}-2- (4-fluorophenyl)-2-[(2- methoxyethyl)(methyl)amino]-N- methylacetamide | 494.46 | 495.2 |

### Examples of Compounds Separated Into Pure Enantiomers or Diastereomers:

One isomer is between 15 to >2000 fold more active than the other isomer.

| | | | | Ret'n | Ret'n | |
|---|---|---|---|---|---|---|
| | | | | time | time | More |
| Ex. | | | Sep. | (min) | (min) | active |
| No. | Structure | Chemical name | Method | isomer 1 | isomer 2 | isomer |
| 3 | 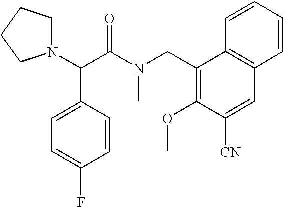 | N-[(3-cyano-2- methoxy-1- naphthyl)methyl]- 2-(4-fluorophenyl)- N-methyl-2- pyrrolidin-1- ylacetamide | 4 | 3.54 | 4.32 | 2 |
| 25 | 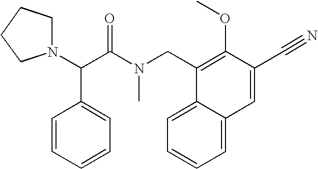 | N-[(3-cyano-2- methoxy-1- naphthyl)methyl]- N-methyl-2-phenyl- 2-pyrrolidin-1- ylacetamide | 26 | 5.91 | 6.64 | 2 |
| 27 | 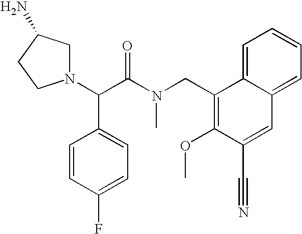 | 2-[(3S)-3- aminopyrrolidin-1- yl]-N-[(3-cyano-2- methoxy-1- naphthyl)-methyl]- 2-(4-fluorophenyl)- N-methylacetamide | 28 | 4.97 | 6.01 | 1 |
| 29 | 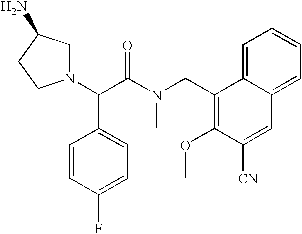 | 2-[(3R)-3- aminopyrrolidin-1- yl]-N-[(3-cyano-2- methoxy-1- naphthyl)methyl]- 2-(4-fluorophenyl)- N-methylacetamide | 30 | 5.07 | 6.17 | 2 |
| 31 | 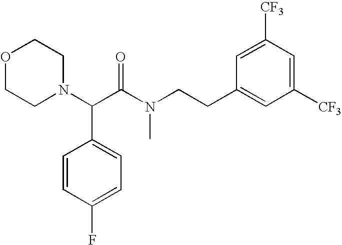 | N-{2-[3,5- bis(trifluoromethyl) phenyl]ethyl}-2-(4- fluorophenyl)-N- methyl-2- morpholin-4- ylacetamide | 32 | 14.42 | 18.82 | 1 |
| 33 | 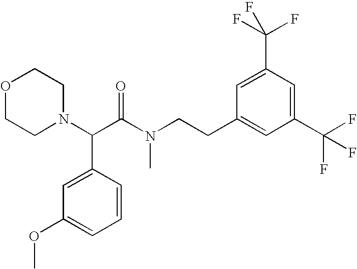 | N-{2-[3,5- bis(trifluoromethyl) phenyl]ethyl}-2-(3- methoxy-phenyl)- N-methyl-2- morpholin-4- ylacetamide | 34 | 16.44 | 18.92 | 1 |
| 35 | 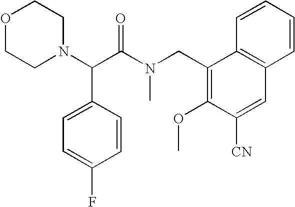 | N-[(3-cyano-2- methoxy-1- naphthyl)methyl]- 2-(4-fluorophenyl)- N-methyl-2- morpholin-4- ylacetamide | 36 | 9.56 | 11.71 | 2 |
| 37 | 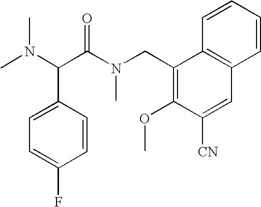 | N-[(3-cyano-2- methoxy-1- naphthyl)methyl]- 2-(dimethylamino)- 2-(4-fluorophenyl)- N-methylacetamide | 38 | 4.86 | 5.41 | 2 |
| 39, 40 | 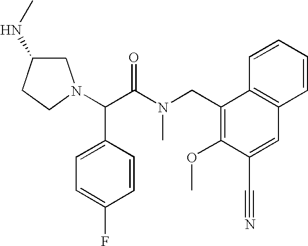 | N-[(3-cyano-2- methoxy-1- naphthyl)methyl]- 2-(4-fluorophenyl)- N-methyl-2-[(3S)- 3-(methylamino)- pyrrolidin-1- yl]acetamide | 39, 40 | 3.62 | 5.33 | 1 |
| 41, 42 | 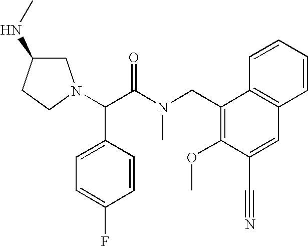 | N-[(3-cyano-2- methoxy-1- naphthyl)methyl]- 2-(4-fluorophenyl)- N-methyl-2-[(3R)- 3-(methylamino)- pyrrolidin-1- yl]acetamide | 41, 42 | 4.61 | 4.43 | 2 |
| 43 | 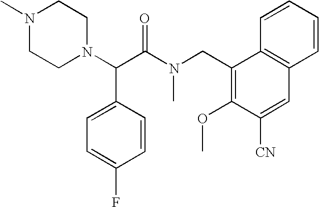 | N-[(3-cyano-2- methoxy-1- naphthyl)methyl]- 2-(4-fluorophenyl)- N-methyl-2-(4- methylpiperazin-1- yl)acetamide | 44, 47 | | | 2 |
| 45 | 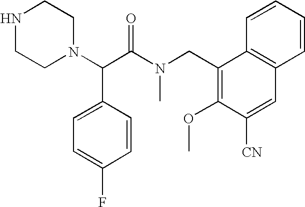 | N-[(3-cyano-2- methoxy-1- naphthyl)methyl]- 2-(4-fluorophenyl)- N-methyl-2- piperazin-1- ylacetamide | 46, 47 | | | 2 |
| 48 | 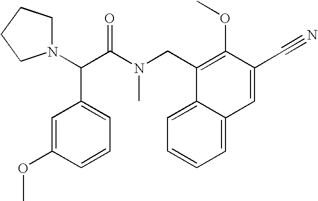 | N-[(3-cyano-2- methoxy-1- naphthyl)methyl]- 2-(3- methoxyphenyl)-N- methyl-2- pyrrolidin-1- ylacetamide | 49 | 9.03 | 12.45 | 2 |

1. A compound in accord with formula I 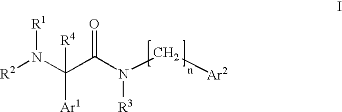
wherein:
R¹ and R² are independently selected from C₁₋₆alkyl or C₁₋₆alkenyl, or together with the N to which they are bound, form a heterocycle having 4, 5, 6, 7 or 8 atoms or such a heterocycle substituted with moieties independently selected from hydrogen, halogen, C₁₋₄alkyl, C₁₋₄alkoxy or C₁₋₄alkyl substituted with 1, 2 or 3 halo moieties, amino, or amino substituted with C₁₋₄alkyl, C₁₋₄alkoxy or C₁₋₄alkyl substituted with 1, 2 or 3 halo moieties;
R³ is C₁₋₆alkyl;
R⁴ is hydrogen;
n is 0, 1 or 2;
Ar¹ is phenyl or phenyl substituted with moieties independently selected from hydrogen, halogen, C₁₋₄alkyl, C₁₋₄alkoxy or C₁₋₄alkyl substituted with 1, 2 or 3 halo moieties, and
Ar² phenyl, naphthyl, tetralin, or phenyl, naphthyl or tetralin substituted with moieties independently selected from hydrogen, halogen, cyano, nitro, C₁₋₄alkyl, C₁₋₄alkoxy or C₁₋₄alkyl substituted with 1, 2 or 3 halo moieties;
in vivo-hydrolysable precursors thereof, and pharmaceutically-acceptable salts thereof.

2. A compound according to claim 1, in accord with formula II 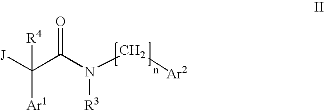
wherein:
J is —NR¹R² or J is selected from moieties of formula III, IV or V, 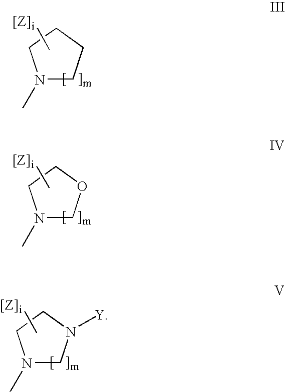
wherein:
when J is —NR¹R²
R¹ and R² are independently selected from H, C₁₋₆alkyl, C₁₋₆alkenyl, C₁₋₆alkanoyl, —CH₂-C(=O)-O-R⁹ or heterocycle,
wherein any such C₁₋₆alkyl, C₁₋₆alkenyl, C₁₋₆alkanoyl, or heterocycle moiety may be substituted with 1, 2 or 3 halo moieties, amino, or amino substituted with C₁₋₄alkyl, C₁₋₄alkoxy or C₁₋₄alkyl substituted with 1, 2 or 3 halo moieties, and R⁹ is selected from hydrogen or C₁₋₆alkyl;
or —(CH₂)ₖX,
where X is selected from —OH, —OR⁵, —C(═O)R⁵ or —NR⁵R⁶ and k is 0, 1, 2, 3 or 4,
wherein R⁵ and R⁶ are independently selected from H. C₁₋₆alkyl, C₁₋₆alkoxy, C₁₋₆alkoxymethylene or C₁₋₆alkenyl,
where any such C₁₋₆alkyl, C₁₋₆alkoxy, C₁₋₆alkoxymethylene or C₁₋₆alkenyl may have 1, 2 or 3 halogen substituents,
or R⁵ and R⁶ together with a N to which they are bound form a heterocycle having 4, 5, 6 or 7 atoms or such a heterocycle substituted with moieties independently selected from halogen, C₁₋₄alkyl, C₁₋₄alkoxy or C₁₋₆alkanoyl, or C₁₋₄alkyl or C₁₋₆alkanoyl substituted with 1, 2 or 3 halo moieties, amino, or amino substituted with C₁₋₄alkyl, C₁₋₄alkoxy or C₁₋₄alkyl, substituted with 0, 1, 2 or 3 halo moieties, and
with the proviso that R¹ and R² are not both hydrogen;
when J is a moiety of formula III, m is 0, 1 or 2;
when J is a moiety of formula IV, m is 2 or 3;
when J is a moiety of formula V, m is 2 or 3 and Y is selected from H, C₁₋₆alkyl, C₁₋₆alkenyl, C₁₋₆alkanoyl or C₁₋₆alkoxycarbonyl where any such C₁₋₆alkyl, C₁₋₆alkenyl, C₁₋₆alkanoyl or C₁₋₆alkoxycarbonyl may have 1, 2 or 3 halogen substituents;
wherein for any moiety of formula III, IV or V, Z is C₁₋₆alkyl, —NR⁷R⁸, or halogen, and i is 0, 1 or 2
wherein R⁷ and R⁸ are independently selected from H, C₁₋₆alkyl C₁₋₆alkenyl or —(CH₂)ₖX, where X is selected from H, —OH, —OR⁵, —C(═O)R⁵ or —NR⁵R⁶,
or R⁷ and R⁸ together with the N to which they are bound, form a moiety of formula VI, VII, VIII or IX, 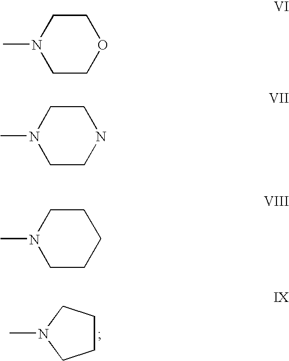
wherein any said moiety of formula VI, VII, VIII or IX may be substituted with 1, 2 or 3 moieties selected from C₁₋₄alkyl, halogen or ═0;
Ar¹ is phenyl or phenyl substituted with moieties independently selected from hydrogen, halogen, C₁₋₄alkyl, C₁₋₄alkoxy or C₁₋₄alkyl substituted with 1, 2 or 3 halo moieties; and
Ar² is phenyl, naphthyl, tetralin, or phenyl, naphthyl or tetralin substituted with moieties independently selected from hydrogen, halogen, cyano, nitro, C₁₋₄alkyl, C₁₋₄alkoxy or C₁₋₄alkyl substituted with 1, 2 or 3 halo moieties;
with the proviso that when J is a moiety of formula V, Ar² is not phenyl,
in vivo-hydrolysable precursors thereof, and pharmaceutically-acceptable salts thereof.

3. A pharmaceutically-acceptable salt of a compound according to claim 1 made with an inorganic or organic acid which affords a physiologically-acceptable anion.

4. A pharmaceutically-acceptable salt of a compound according to claim 3, wherein said inorganic or organic acid is selected from hydrochloric, hydrobromic, sulfuric, phosphoric, methanesulfonic, sulfamic, para-toluenesulfonic, acetic, citric, lactic, tartaric, malonic, fumaric, ethanesulfonic, benzenesulfonic, cyclohexylsulfamic, salicyclic and quinic acids.

5. A pharmaceutical composition comprising a compound according to claim 1, an in vivo-hydrolysable precursor or a pharmaceutically-acceptable salt thereof and a pharmaceutically-acceptable carrier.

6. A method of treating a disease condition wherein antagonism of NK₁ receptors is beneficial which method comprises administering to a warm-blooded animal an effective amount of a compound according to claim 1 or an in vivo-hydrolysable precursor or a pharmaceutically-acceptable salt thereof.

7. A method of treating a disease condition wherein antagonism of NK₁ receptors is beneficial which method comprises administering to a warm-blooded animal an effective amount of a compound according to claim 1 or an in vivo-hydrolysable precursor or a pharmaceutically-acceptable salt thereof.

8-9. (canceled)

10. A method for treating a disorder or condition selected from depression in cancer patients, depression in Parkinson's patients, postmyocardial infarction depression, subsyndromal symptomatic depression, depression in infertile women, pediatric depression, major depression, single episode depression, recurrent depression, child-abuse induced depression, post-partum depression, generalized anxiety disorder, agoraphobia, social phobia, simple phobias, posttraumatic stress syndrome, avoidant personality disorder, obsessive-compulsive disorder, panic disorder, dementia, hyperprolactinaemia, cerebellar ataxia, gastrointestinal tract disorders, negative symptoms of schizophrenia, premenstrual syndrome and stress incontinence in a mammal, wherein antagonism of the NK₁ receptors is beneficial, comprising administering an effective amount of a compound according to claim 1 or a pharmaceutically-acceptable salt thereof effective in treating such disorder or condition.

11. The method according to claim 10, wherein said compound is administered in combination with a pharmaceutically-acceptable carrier.

12. The method according to claim 6, wherein said compound is administered in combination with a pharmaceutically-acceptable carrier.

13. The method according to claim 7, wherein said compound is administered in combination with a pharmaceutically-acceptable carrier.
